(19) United States
(12) Patent Application Publication
(10) Pub. No.: US 2025/0339215 A1
(43) Pub. Date: Nov. 6, 2025

(54) SYSTEMS, DEVICES, AND METHODS EMPLOYING A CARTRIDGE FOR SURGICAL TOOL EXCHANGE IN A SURGICAL ROBOTIC SYSTEM

(71) Applicant: Vicarious Surgical Inc., Waltham, MA (US)

(72) Inventors: Joseph Peine, Somerville, MA (US); Andrew Degonge, Cambridge, MA (US); Maeve Devlin, East Boston, MA (US); Tyler McCollum, Somerville, MA (US)

(21) Appl. No.: 18/869,770

(22) PCT Filed: Jun. 1, 2023
(86) PCT No.: PCT/US2023/024164

§ 371 Date: Nov. 26, 2024

Related U.S. Application Data
(60) Provisional application No. 63/347,958, filed on Jun. 1, 2022.

(51) Int. Cl. A61B 34/30; A61B 17/00; A61B 90/98; B25J 15/04

(52) U.S. Cl. CPC A61B 34/30; A61B 90/98; B25J 15/04; A61B 2017/00477

(57) ABSTRACT

Systems and methods employing cartridges for securing one or more tool elements of a surgical tool and releasing the tool elements when engaged by a distal end of a robotic arm are provided. A cartridge includes a cartridge body, a holder disposed in a cartridge body and configured to engage tool elements, a spring connecting the holder to the cartridge body, and a pair of biasing elements each attached to the cartridge body at a first end and having a retention mechanism at a second end, the retention mechanism configured to engage corresponding receiving mechanisms of tool elements. Also provided is a cartridge with an RFID tag and/or RFID tag and RFID blocking flag systems to identify proximity of a distal end of a robotic arm.

### RELATED APPLICATIONS

The present application claims the benefit of U.S. Provisional Application Ser. No. 63/347,958 filed on Jun. 1, 2022, the contents of which are incorporated by reference herein.

### BACKGROUND OF THE DISCLOSURE

Surgical robotic systems may use manipulators, graspers, scalpels, scissors, cautery devices, and other tools that are inserted into a patient through a single incision or multiple incisions, such as through a cannula, for laparoscopic procedures. In some procedures, a variety of tools and graspers are needed to complete a surgical procedure. The tools may be interchangeable tools that are mounted to the surgical robotic systems, for example, by being mounted to an arm of the surgical robotic system. In some cases, the tools may be mounted to and removed from the arm before or during a procedure.

### SUMMARY OF THE INVENTION

The present disclosure is directed to a cartridge for securing one or more tool elements of a surgical tool and releasing the tool elements when engaged by a distal end of a robotic arm. The cartridge may include a cartridge body defining a tool holding cavity, a holder channel, an arm and tool channel, and an access aperture, the arm and tool channel extending from the tool holding cavity to the aperture on a first side of the cartridge body and configured to receive a distal end of a robotic arm. The cartridge may include a holder within the tool holding cavity configured to releasably secure the one or more tool elements.

The present disclosure is directed to a cartridge for securing one or more tool elements of a surgical tool and releasing the tool elements when engaged by a distal end of a robotic arm. The cartridge includes a cartridge body including an arm and tool channel extending to an opening on a first side of the cartridge body and configured to receive a distal end of a robotic arm, and including a holder channel. The cartridge also includes a holder including; a cradle portion configured to engage at least a portion of the one or more tool elements; and an end portion opposite the cradle portion. The end portion of the holder extends into the holder channel. The cartridge also includes a spring disposed in the holder channel. The spring has a first end connected to the end portion of the holder and has a second end connected to the cartridge body. The spring and holder channel s configured to permit movement of the holder relative to the cartridge body upon compression or extension of the spring. The holder also includes a pair of biasing elements, each biasing element including a retention mechanism disposed at a proximal end of the biasing element and each biasing element secured to the cartridge body at a distal end. Each retention mechanism is configured to engage a corresponding receiving mechanism in a tool element body of a corresponding tool element. The retention mechanism may be any feature to engage with the corresponding receiving mechanism. The retention mechanism may be, for example, a detent or other protruding feature or may be a magnet. The receiving mechanism may be any feature to engage with the corresponding retention mechanism. The receiving mechanism may be, for example, a notch, indent, semi-spherical recess, or other recessed feature, or may be a magnet.

In some embodiments, the holder defines a pair of biasing element channels, each of the pair of biasing elements extending through a corresponding one of the biasing element channels. The biasing element channels are configured to enable the holder to move along a central axis of the holder channel with respect to the pair of biasing elements while restricting lateral deflection of a portion of the biasing elements positioned within the biasing element channels.

In some embodiments, the pair of biasing element channels are configured to hold the proximal ends of the biasing elements in a first position when the spring is in an extended position and to enable the proximal ends of the biasing elements to flex outward when the spring is in a compressed position.

In some embodiments, the retention mechanism has a shape of any of a ball, a latch, a hook, or a protrusion. Some embodiments further include a surgical tool, which includes tool elements, held by the cradle portion of the holder. In some embodiments, the surgical tool is selected from the group consisting of graspers, bipolar forceps, scissors, and needle drivers. In embodiments, a first force against the surgical tool in a first direction causes the holder to shift in the first direction. In some embodiments, the opening of the cartridge for insertion of the distal end of the robotic arm is covered, at least in part, by a covering and the covering is a door, a flap, or a single-use sheet. In some embodiments, the surgical tool held by the cradle portion is accessible to a distal end of a robotic arm passing through the opening of the cartridge. In some embodiments, the holder includes a protrusion configured to engage with a groove in a tool. The protrusion may be a cylindrical protrusion.

The present disclosure is directed to a surgical robotic system. The surgical robotic system includes a cartridge as described herein and a robotic arm including a distal end configured to hold a surgical tool. In some embodiments, the holder is configured to be moved in a first direction by a force applied by the robotic arm in the first direction.

The present disclosure is directed to a method for providing a surgical tool to a robotic surgical system including a robotic arm. The method includes: providing a cartridge including a surgical tool held in a cradle portion of a holder of the cartridge; inserting a distal end of the robotic arm into the cartridge; engaging the surgical tool with the distal end of the robotic arm; shifting the holder of the cartridge and actuating the surgical tool to release the surgical tool from the cartridge; and withdrawing the distal end of the robotic arm with the surgical tool mounted on the thereon from the cartridge.

In some embodiments, actuating the surgical tool to release the surgical tool from the cartridge includes: applying a force via the distal end of the robotic arm against the holder in a first direction; and moving the holder in the first direction releasing a portion of at least one biasing element held in part by the holder and actuating the surgical tool to disengages the at least one biasing element from the surgical tool. In some embodiments, the first direction is a direction along an axis of the cartridge from an opening of the cartridge to a cartridge side opposite the opening.

The present disclosure is directed to a method for providing a surgical tool to a surgical robotic system. The method may include providing a cartridge holding a surgical tool within a holder of the cartridge. The method may include inserting a distal end of a robotic arm of the surgical robotic system into the cartridge. The method may include engaging the surgical tool using the distal end of the robotic arm. The method may include releasing the surgical tool from the holder. The method may include withdrawing the surgical tool from the cartridge. The method may include releasing the surgical tool from the holder comprises actuating tool elements of the surgical tool to flex retention mechanisms (e.g., detents) of biasing elements of the holder of the cartridge away from corresponding receiving mechanisms (e.g., notches) in the surgical tool. The method may include withdrawing the surgical tool comprises rotating or closing the surgical tool via the robotic arm to release the surgical tool from the holder of the cartridge. The method may include pivoting a cartridge lever of the cartridge in order to allow access to the cartridge by the robotic arm.

The present disclosure is directed to a cartridge including an RFID tag. The cartridge may also include an RFID blocking flag moveable between a blocking position and a non-blocking position; and at least one mechanical linkage connected to the RFID blocking flag to shift the RFID blocking flag between the blocking position and the non-blocking position.

In one aspect, the present disclosure is directed to a cartridge for holding and/or storing surgical tools for a robotic surgical system, the cartridge including: a cartridge body with an opening therein; a holder including a cradle portion configured to hold a surgical tool; an RFID tag; and an RFID blocking flag; and at least one mechanical linkage permitting at least in part a mechanical communication between the RFID blocking flag and the holder.

In some embodiments, the RFID blocking flag is at least substantially impervious to an RFID signal. In some embodiments, the RFID blocking flag is moveable between a first position and a second position, and the RFID blocking flag in the first position blocks a signal to and from the RFID tag and in the second position permits a signal to and from the RFID tag.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a schematic illustration of an exemplary surgical robotic system that implements the robot end effectors in accordance with some embodiments.

FIGS. 2A and 2B are perspective views of a robotic arm of a robotic unit in accordance with some embodiments.

FIGS. 3 and 4 are exploded perspective views of the end effector portion of the robotic arm of FIGS. 2A and 2B in accordance with some embodiments.

FIGS. 5A, 5B, 5C, and 5D are perspective views of a surgical tool for use in a surgical robotic system engaged with a distal end of a robotic arm of the surgical robotic system in accordance with some embodiments.

FIG. 6 is top view of a surgical tool for use in a surgical robotic system and suitable for use in an embodiment of a cartridge as described herein in accordance with some embodiments.

FIG. 7A a top view of a tool element of a surgical tool for use in a surgical robotic system and suitable for use in some embodiments of cartridges described herein.

FIGS. 7B-7C are a perspective view and a top view, respectively of a tool element of a surgical tool and suitable for use in suitable for use in some embodiments of cartridges described herein.

FIG. 8A is a perspective view of a cartridge holding tool elements in accordance with some embodiments

FIG. 8B is a top cross-sectional view of the cartridge of FIG. 8A and a distal end of a robotic arm in accordance with some embodiments.

FIG. 9A is a perspective view of a cartridge with a surgical tool in which the cover portion of the cartridge body is omitted for illustrative purposes in accordance with some embodiments.

FIG. 9B is a side view of a cartridge with a surgical tool of FIG. 9A.

FIGS. 10A, 10B, and 10C are top cross-sectional views of insertion of a distal end of a robotic arm into the cartridge of FIGS. 8A and 8B cartridge in accordance with some embodiments.

FIGS. 11A and 11B are top cross-sectional views and FIGS. 11C and 11D are perspective views of a distal end of a robotic arm and tool elements stored in a cartridge of

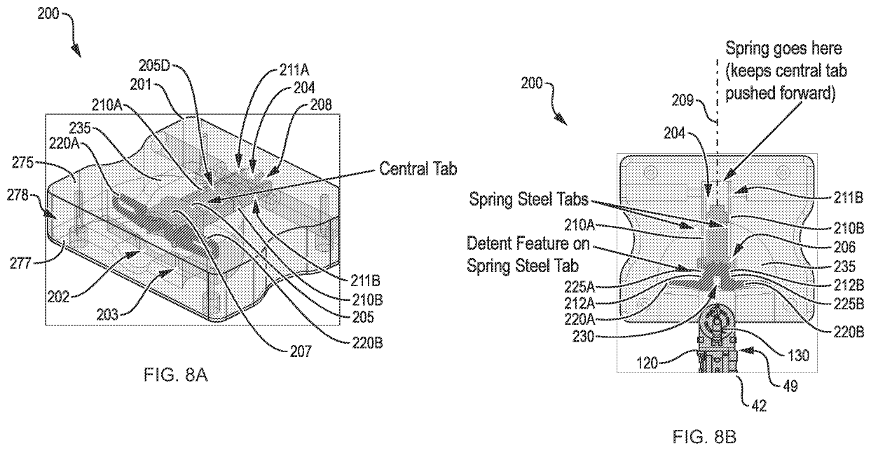

FIGS. 8A and 8B cartridge in accordance with some embodiments.

FIGS. 12A and 12B are top cross-sectional views and FIGS. 12C and 12D are perspective views of a distal end of the robotic arm and the cartridge with the surgical tool elements and a distal end of a robotic arm in an engaged state in accordance with some embodiments.

FIGS. 13A and 13B are top cross-sectional views and FIGS. 13C and 13D are perspective views of the distal end of the robotic arm and the cartridge in an engaged state and the arm actuated in accordance with some embodiments.

FIGS. 14A, 14B, 14C, and 14D are views of an embodiment of a cartridge and engaged tool elements.

FIG. 14E is a perspective view of a distal end of a robotic arm engaged with and actuating tool elements in the cartridge of FIGS. 14A-14C depicts an embodiment of a cartridge according to the present disclosure with a surgical tool and a distal end of a robotic arm.

FIG. 15A is a schematic view of an embodiment of a cartridge in a stored state.

FIG. 15B is a schematic view of the cartridge with a distal end of a robotic arm in an engaged and activated state.

FIG. 16 is a flowchart illustrating a method for securing one or more tool elements of a surgical tool within a cartridge and releasing the tool elements when engaged by s distal end of a robotic arm in accordance with some embodiments.

FIGS. 17A, 17B, 17C, 17D, and 17E are views of the cartridge with the cartridge lever in a closed position according to some embodiments

FIGS. 18A, 18B, 18C, 18D, and 18E are views of the cartridge with the cartridge lever in an open position according to some embodiments.

FIG. 19 is a perspective view of a cross-section of the cartridge with the cartridge lever and the tool positioned therein according to some embodiments with the cartridge lever in the closed position.

FIG. 20 is a perspective view of the holder with the tool according to some embodiments.

FIG. 21A is a top cross-sectional view showing the cartridge with the cartridge lever in an open position and a distal end of the arm of the surgical robotic system connected to the tool.

FIG. 21B is a top cross-sectional view showing the cartridge with the cartridge lever in a closed position and a distal end of the arm of the surgical robotic system connected to the tool.

FIGS. 21C and 21D are top cross-sectional views showing the cartridge with the cartridge lever in an open position and the distal position of the arm of the surgical robotic system partially withdrawn from the cartridge.

FIGS. 22A, 22B, 22C, and 22D are views of the cartridge with release mechanism according to some embodiments.

FIGS. 23A, 23B, and 23C are views of a portion of the release mechanism according to some embodiments.

FIGS. 24A, 24B, and 24C are views of a portion of the cartridge showing the spring and the holder and a portion of the robotic arm according to some embodiments.

FIGS. 25A, 25B, 25C, and 25D are views of a portion of the cartridge showing the spring and the holder and a portion of the robotic arm according to some embodiments.

FIGS. 26A, 26B, 26C, and 26D are views of a portion of the cartridge showing the spring and the holder and a portion of the robotic arm according to some embodiments.

FIGS. 27A, 27B, and 27C are views of a portion of the cartridge showing the spring and the holder and a portion of the robotic arm according to some embodiments.

FIG. 28 is a view of a portion of the cartridge showing the spring and the holder and a portion of the robotic arm according to some embodiments.

FIG. 29 is a view of a portion of a cartridge and a portion of a distal end of a robotic arm including an RFID antenna.

FIG. 30 is a view of a portion of a cartridge featuring an RFID blocking flag in a blocking position and a portion of a distal end of a robotic arm including an RFID antenna.

FIG. 31 is a view of a portion of a cartridge featuring an RFID blocking flag in a non-blocking position and a portion of a distal end of a robotic arm including an RFID antenna.

### DETAILED DESCRIPTION

Embodiments taught herein provide cartridges for tools of surgical robotic devices and methods of using the same to attach a tool to a surgical robotic device, such as to an arm of a surgical robotic device.

Embodiments of cartridges taught herein can provide a number of advantages. Embodiments of the cartridges taught herein may provide systems and devices to provide tools for use in the surgical robotic system in a cartridge that is manipulable by a user to facilitate selecting and attaching the tool. Embodiments may provide a cartridge that is ergonomic and configured to be conveniently held by a user such that the user can maintain a firm grasp on the cartridge to better engage the cartridge and/or tool with the robotic surgical system. The shape of the cartridge in some embodiment may help to guide an arm of the surgical robotic system to facilitate attaching the tool to the arm. Embodiments may provide a sterile environment for the tool and may secure the tool within the cartridge in a manner that reduces or eliminates potential for exposure of the user to the tool reducing a possibility for contamination or for injury to the user (e.g., by a user inadvertently being cut or pinched by a tool). Embodiments may also provide flexibility in how the surgical robotic device is operated. For example, initially inserting all of the necessary tools to be employed by the surgical robotic system at once within the patient can result in an increased risk to the patient due to the possible use of excess incision sites and the inherent increased complexity of safely storing and manipulating the tools during the surgical procedure. The present technology may facilitate removing and replacing tools throughout the surgery in a safe and convenient manner. After a procedure, tools may be replaced in the cartridges provided herein for disposal of the tool in a sanitary manner or for cleaning and reuse of the tool.

While various embodiments of devices, systems, and methods for a cartridge for surgical tool exchange in a surgical robotic system are illustrated and described herein, it will be clear to those skilled in the art that such embodiments are provided by way of example. It will be apparent to one skilled in the art, however, that the disclosed subject matter may be practiced without such specific details, and that certain features, which are well known in the art, are not described in detail in order to avoid complication and enhance clarity of the disclosed subject matter. In addition, it will be understood that any examples provided below are merely illustrative and are not to be construed in a limiting manner, and that it is contemplated by the present inventors that other systems, apparatuses, and/or methods can be employed to implement or complement the teachings of the present invention and are deemed to be within the scope of the present invention. For convenience, like reference numbers are used to reference similar features of the various embodiments shown in the figures, unless otherwise noted.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some exemplary embodiments may be described herein or in documents incorporated by reference as employing a plurality of units to perform exemplary processes, it is understood that exemplary processes may also be performed by one or a plurality of modules. Additionally, it is understood that the term controller/control unit may refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or control units or multiple different types of controllers or control units may be employed in performing one or more processes. In some embodiments, different controllers or control units may be implemented in different portions of a surgical robotic system.

While the system and method of the present invention can be designed for use with one or more surgical robotic systems employed as part of a virtual reality surgical system, the robotic system of the present invention may be employed in connection with any type of surgical system, including for example robotic surgical systems, straight-stick type surgical systems, and laparoscopic systems. Additionally, the system of the present invention may be used in other non-surgical systems, where a user requires access to a myriad of information, while controlling a device or apparatus.

The present disclosure provides systems, devices and methods employing a cartridge for secure surgical tool exchange in a surgical robotic system. In some embodiments, the cartridges may include a cartridge body with an opening therein; a holder comprising a cradle portion configured to hold a surgical tool; a spring connected to the holder and cartridge body and configured to permit movement of the holder relative to the cartridge body upon compression and extension of the spring; and a pair of biasing elements each comprising a retention mechanism at a first end configured to engage a corresponding receiving mechanism on a tool element and a second end attached to the cartridge body. In some embodiments, the holder also includes a pair of biasing element channels of biasing element channels, each of the pair of biasing elements extending through a corresponding one of the biasing element channels, where the biasing element channels configured to enable the holder to move along an axis a central axis of a holder channel of the cartridge body with respect pair of biasing elements while restricting lateral deflection of a portion of the biasing elements positioned within the biasing element channels. Insertion of a robotic arm and force of the robotic arm against the holder causes the holder to move along the central axis, permitting the retention mechanisms of the biasing elements to deflect and disengage from the receiving mechanism of the tool elements, thereby releasing the tool elements after they are engaged with the robotic arms.

Also provided are cartridges with RFID tag or chips configured to be read by a distal end of a robotic arm when the robotic arm is inserted into the cartridge for installation or removal of tool elements. In some embodiments, a cartridge also includes an RFID blocking flag that selectively reduces a signal from an RFID tag or chip to enable greater accuracy in using an RFID signal to detect the proximity of a robotic arm to the RFID tag or chip of the cartridge.

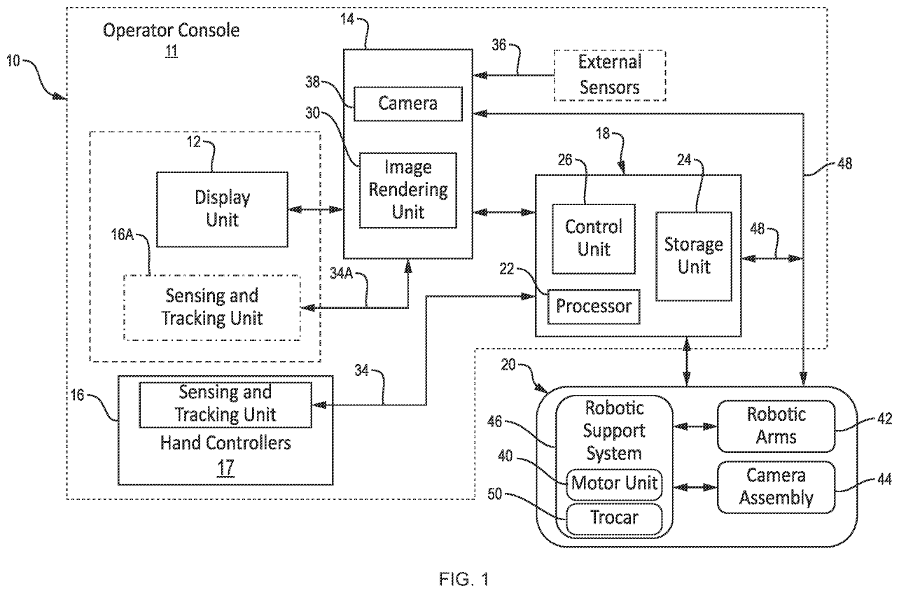

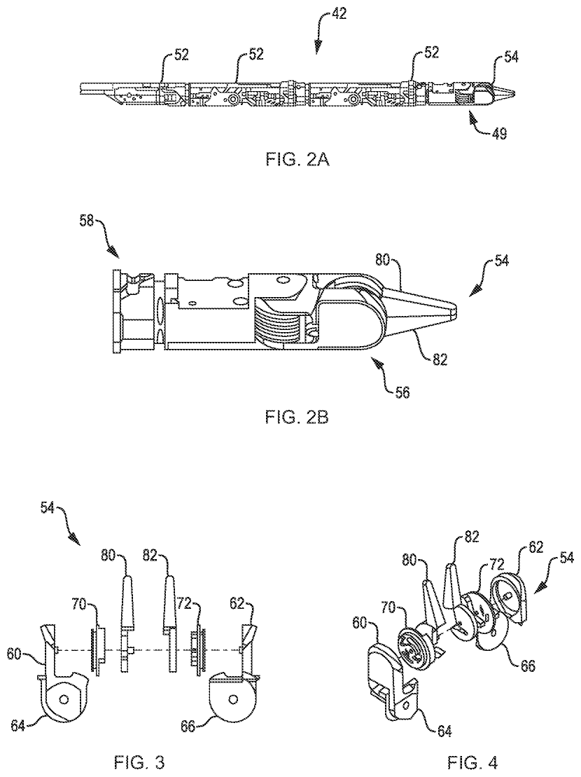

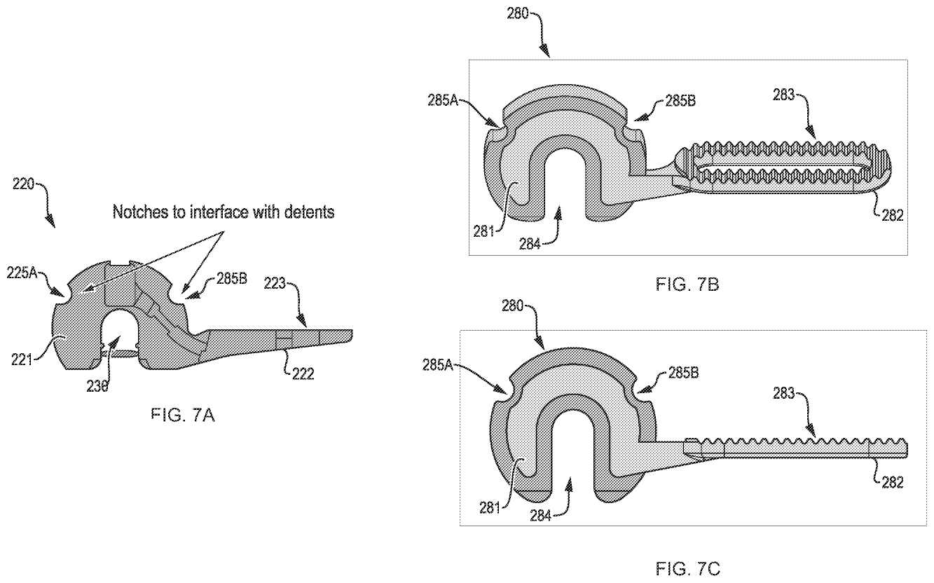

Prior to describing tool elements, surgical tool exchange, and cassettes for surgical tool exchange with respect to FIGS. 7A-31, aspects of surgical robotic systems in which some embodiments may be employed are described below and in connection with FIG. 1, and aspects of a robotic arms and end effectors in which some embodiments may be employed are described below with respect to FIGS. 2-6.

### Surgical Robotic Systems and End Effectors

A system for robotic surgery may include a robotic subsystem that includes a surgical robotic unit that can be inserted into a patient via a trocar through a single incision point or site. The robotic unit is small enough to be deployed in vivo at the surgical site and is sufficiently maneuverable when inserted to be able to move within the body to perform various surgical procedures at multiple different points or sites. The surgical robotic unit includes multiple separate robotic arms that are deployable within the patient along different or separate axes. Further, a surgical camera assembly can also be deployed along a separate axis. Thus, the surgical robotic unit employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor unit and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic unit, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display device or unit 12, an image computing unit 14, which may be a virtual reality (VR) computing unit, hand controllers 17 having a sensing and tracking unit 16, and a computing unit 18.

The display unit 12 can be any selected type of display for displaying information, images or video generated by the image computing unit 14, the computing unit 18, and/or the robotic subsystem 20. The display unit 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display unit 12 can also include an optional sensing and tracking unit 16A. In some embodiments, the display unit 12 can include an image display for outputting an image from a camera assembly 44 of the robotic subsystem 20.

In some embodiments, if the display unit 12 includes an HMD device, an AR device that senses head position, or another device that employs an associated sensing and tracking unit 16A, the HMD device or head tracking device generates tracking and position data 34A that is received and processed by image computing unit 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensing and tracking unit 16A to provide position and orientation tracking of the operator's head. The sensing and tracking unit 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensing and tracking unit 16A, can track the position and orientation of the operator's head, generate tracking and position data 34A, and then relay the tracking and position data 34A to the image computing unit 14 and/or the computing unit 18 either directly or via the image computing unit 14.

The hand controllers 17 are configured to sense a movement of the operator's hands and/or arms to manipulate the surgical robotic system 10. The hand controllers 17 can include the sensing and tracking unit 16, circuity, and/or other hardware. The sensing and tracking unit 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensing and tracking unit 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracking unit 16A as described above. In some embodiments, the sensing and tracking unit 16 can be external and coupled to the hand controllers 17 via electricity components and/or mounting hardware.

In some embodiments, the sensing and tracking unit 16 can employ sensors coupled to the torso of the operator or any other body part. In some embodiments, the sensing and tracking unit 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking unit 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors can generate external data 36 that can be processed by the computing unit 18 and hence employed by the surgical robotic system 10.

The sensors generate position and/or orientation data indicative of the position and/or orientation of the operator's hands and/or arms. The sensing and tracking units 16 and/or 16A can be utilized to control movement (e.g., changing a position and/or an orientation) of the camera assembly 44 and robotic arms 42 of the robotic subsystem 20. The tracking and position data 34 generated by the sensing and tracking unit 16 can be conveyed to the computing unit 18 for processing by at least one processor 22.

The computing unit 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage unit 24. The tracking and position data 34A can also be used by the control unit 26, which in response can generate control signals for controlling movement of the robotic arms 42 and/or the camera assembly 44. For example, the control unit 26 can change a position and/or an orientation of at least a portion of the camera assembly 44, of at least a portion of the robotic arms 42, or both. In some embodiments, the control unit 26 can also adjust the pan and tilt of the camera assembly 44 to follow the movement of the operator's head.

The robotic subsystem 20 can include a robot support system (RSS) 46 having a motor unit 40 and a trocar 50, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor unit 40 and the trocar 50. The RSS 46 can further include a support member that supports the motor unit 40 coupled to a distal end thereof. The motor unit 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor unit 40 that is coupled to the robotic subsystem 20 at one end and to an adjustable support member or element at an opposed end.

The motor unit 40 can receive the control signals generated by the control unit 26. The motor unit 40 can include gears, one or more motors, drivetrains, electronics, and the like, for powering and driving the robotic arms 42 and the cameras assembly 44 separately or together. The motor unit 40 can also provide mechanical power, electrical power, mechanical communication, and electrical communication to the robotic arms 42, the camera assembly 44, and/or other components of the RSS 46 and robotic subsystem 20. The motor unit 40 can be controlled by the computing unit 18. The motor unit 40 can thus generate signals for controlling one or more motors that in turn can control and drive the robotic arms 42, including for example the position and orientation of each articulating joint of each robotic arm, as well as the camera assembly 44. The motor unit 40 can further provide for a translational or linear degree of freedom that is first utilized to insert and remove each component of the robotic subsystem 20 through the trocar 50. The motor unit 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. The robotic subsystem 20 can be supported by the trocar with multiple degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking unit 16, the robotic arms 42, the camera assembly 44, and the like), and for generating control signals in response thereto. The motor unit 40 can also include a storage element for storing data.

The robotic arms 42 can be controlled to follow the scaled-down movement or motion of the operator's arms and/or hands as sensed by the associated sensors. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms of the robotic arms 42 may follow movement of the arms of the operator in some modes of control, the robotic shoulders are fixed in position in such modes of control. In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display unit 12. In embodiments in which the display unit 12 includes a HMD, the display can include the built-in sensing and tracking unit 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking unit. In some embodiments, the sensing and tracking unit 16A may be used to provide supplementary position and orientation tracking data of the display in lieu of or in addition to the built-in tracking system of the HMD. In some embodiments, no head tracking of the operator is used or employed.

FIGS. 2A and 2B illustrate the general design of selected components of a robotic arm 42 that enable the user or operator to replace tool elements of the robotic arm 42 without requiring the replacement of the entire robotic arm 42 in accordance with some embodiments. As such, the end effector region of the robotic arms provide for a highly functional, easy to use, mechanical connection that allows for the easy removal and replacement of tools in some embodiments. Cartridges that further facilitate secure tool element exchange in accordance with some embodiments and tool elements configured to be held by such cartridges are described below with respect to FIGS. 7A-27.

For the sake of simplicity, only a single robotic arm 42 is shown in FIGS. 2A and 2B, although a second robotic arm or subsequent robotic arms can be similar or identical in form and function, or may have a different form. A robotic arm may also or alternatively be referred to as a “robot arm” herein. The illustrated robotic arm 42 can include a series of articulation segments 52 that form joint sections that correspond to the joints of a human arm in accordance with some embodiments. As such, the articulation segments 52 can be constructed and combined to provide for rotational and/or hinged movement to emulate different portions of the human arm, such as for example the shoulder joint or region, elbow joint or region, and the wrist joint or region 58. The articulation segments 52 of the robotic arm 42 are constructed to provide cable-driven, rotational movement, for example, but within the confines of reasonable rotational limits in accordance with some embodiments. The articulation segments 52 are configured to provide maximum torque and speed with minimum size in accordance with some embodiments. The articulation segments 52 are mechanically coupled and end in an end effector portion or segment 54. The end effector portion 54 includes a tool base portion 56 that can incorporate therein any selected surgical tool or tool elements to be employed to perform a desired or selected surgery in some embodiments. For example, the depicted tool base portion 56 mounts a pair of tool elements 80, 82. In the current example, the tool elements are grippers, although those of ordinary skill in the art in view of the present disclosure will readily recognize that any selected type of surgical tool can be employed, for example, any of graspers, bipolar forceps, scissors, needle drivers, and cautery hooks. As shown in FIG. 2B, the end effector portion 54 and the adjacent articulation segment 52 form a wrist portion or joint 58 of the robotic arm in some embodiments. The end effector portion 54 is also shown in detail in FIGS. 3-12C in accordance with some embodiments.

As shown in FIGS. 2A and 2B, the end effector portion 54 includes opposed tool base segments 60 and 62, pulley elements 70 and 72, and tool elements 80 and 82, shown as a pair of gripper or grasper elements in accordance with some embodiments. The tool base portion 56 is assembled by mounting the pulley element 70 to the tool base segment 60 via a protrusion, such as a post in some embodiments. Likewise, the pulley element 72 is mounted to the tool base segment 62 via a similar post in some embodiments.

As illustrated, the tool base portion 56 can include two independently driven, rotating pulley elements 70, 72. When the pulley elements are disposed so as to open the tool elements into a wider angle than is needed in surgery (e.g., an open tool exchange position), mechanical features on the pulley elements align and allow the tool elements to be easily removable therefrom, such as by sliding off, or by gently pushing the tool off of the tool base depending on the selected open geometry in accordance with some embodiments.

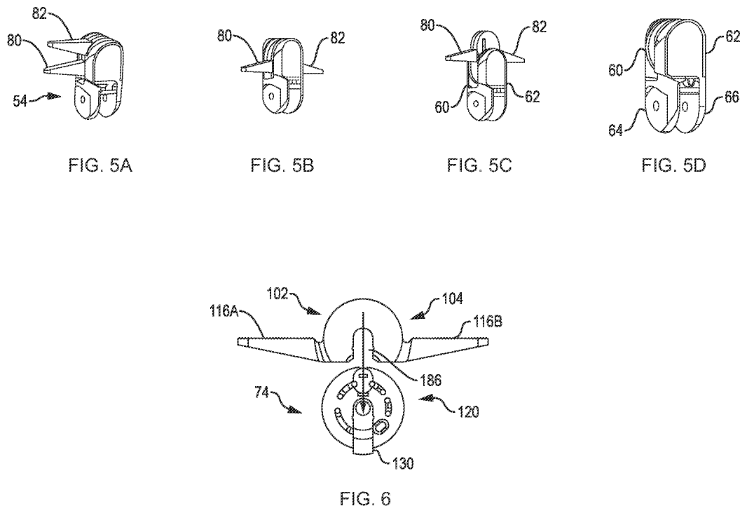

FIGS. 5A to 5D show certain features of an embodiment of end effector portion or segment 54 featuring tool elements 80 and 82, opposed tool base segments 60 and 62, and connecting flanges 64 and 66. As shown in FIGS. 5A to 5D, opposed tool elements 80, 82 may be rotated with respect to opposed tool base segments 60 and 62 to achieve different positions during a procedure and to permit cutting or other operations. FIG. 6 shows an end effector portion or segment 74 with knurled or serrated working surfaces 116A and 116B on tool elements 102 and 104. As illustrated in FIG. 6, tool elements 102 and 104 define a slot 186. FIG. 6 shows the engagement of end effector portion or segment 74 with tool elements 102 and 104 and raised or protruding boss element 130 which form a portion of the distal end 49 of a robotic arm, such as arm 42 of surgical robotic unit 50 as shown on FIG. 2A. Raised or protruding boss element 130 and pulley element 120 are configured to mate with slot 186 of end effector portion or segment 74.

### Tools

In some aspects, systems, devices, and methods disclosed herein employ cartridges that hold tool elements with corresponding mating features and facilitate installing the tool elements on a distal end of a robotic arm to form a functional end effector tool. The cartridges also function to receive and hold tool elements from the distal end of the robotic arm enabling different tool elements held in a different cartridge to be installed on the same robotic arm. In embodiments, the cartridge is designed to securely hold tool elements to permit easy retrieval of the tool elements and installation on a distal end of a robotic arm forming a functional tool for use in a surgical procedure. Tools contemplated herein include, but are not limited to, graspers, bipolar forceps, scissors, needle drivers, and cautery hooks. The tools may also be configured to include conductive contact elements. The cartridges of the present invention may feature internal structures designed to hold a tool elements within a tool holding cavity of the cartridge to permit retrieval of the tool elements by a robotic arm. Because the tool elements are held within the cartridge, the tool elements may be retained in a sanitary and/or sterile condition and may be protected from damage due to being dropped, impacted by other objects, or other contact. Additionally, the use of the cartridge system may facilitate substituting tool elements corresponding to different tools, and conveniently permit the use of multiple tools by the robotic arms of the robotic system during a surgical procedure. Further, the use of the cartridges may facilitate shipping, storage and inventory for the surgical tool elements.

FIG. 7A shows a tool element 220 configured to be held by a cartridge 200 in accordance with some embodiments. Tool element 220 includes a base portion 221 that defines a slot 230 and includes notches 225A, 225B as receiving mechanisms. Tool element also includes an extension portion 222 extending from the base portion. In some embodiments, the extension portion 222 has a smooth gripping surface 223. FIGS. 7B and 7C show views of another embodiment of a tool element 280. Tool element 280 includes a base portion 281 that defines a slot 284 and includes notches 285A, 285B. Tool element 280 also has an extension portion 282 extending from the base portion 281. The extension portion 282 includes a serrated gripping surface 283 in accordance with some embodiments.

### Cartridges

FIGS. 8A and 8B illustrate a cartridge 200 according to some embodiments of the present disclosure. The cartridge 200 includes a cartridge body 201. The cartridge body 201 includes a cover portion 275 joined to a base portion 277. The cartridge body 201 defines a tool holding cavity 235, a holder channel 204, an arm and tool channel 202, and an access aperture 203. The cartridge 200 includes a holder 205 disposed within the cartridge body 201 in some embodiments. The holder 205 includes a cradle portion 207 configured to engage at least a portion of the tool elements. In some embodiments, cradle portion 207 includes an upper cradle portion part and a lower cradle portion part for engaging and holding tool elements 220A, 220B. A second end portion of the holder 205D opposite the cradle portion extends into a holder channel 204 of the cartridge body and is connected via a spring 208 to cartridge body 201 either directly or through one or more different elements. The spring 208 and holder channel 204 are configured to permit movement of the holder 205 along a central axis 209 of the holder channel 204 relative to the cartridge body 201 upon compression or extension of the spring 208.

The cartridge 200 also includes biasing elements 210A, 210B extending parallel to a central axis 209 of the holder channel 204. In some embodiments, biasing elements 210A, 210B may be spring tabs. In some embodiments, each biasing element includes detents 212A, 212B as retention mechanisms at a first end of the biasing element, which may be referred to as a proximal end of the biasing element. Each detent is configured to engage by a corresponding notch on a tool element. Each biasing element 210A, 210B is connected to the cartridge body at second end 211A, 211B, which may be described as a distal end in some embodiments. Holder 205 includes biasing element channels 206 through which the biasing elements 210A, 210B extend. The biasing element channels 206 are configured to enable the holder 205 to move along an axis a central axis 209 of the holder channel 209 with respect to the pair of biasing elements 210A, 210B while restricting lateral deflection of a portion of the biasing elements positioned within the biasing element channels 206.

The cartridge body 201 includes an arm and tool channel 202 extending to an opening (access aperture 203) on a side 278 of the cartridge body 201. The arm and tool channel 202 is configured to receive a distal end 49 of a robotic arm 42, to permit rotation of tool elements (e.g., tool elements 220A, 220B), and to permit withdrawal of the distal end 49 of the robotic arm 42 with the tool elements 220A, 220B installed on the robotic arm 42.

At least a portion of the cartridge body 201, for example, at least a portion of the cover portion 275 of the cartridge body 201 may be formed of a transparent material permitting visual identification of, or confirmation of the presence and/or configuration of the tool elements 220A, 220B within the cartridge 200.

FIG. 8B shows the detents 212A, 212B engaged with the notches 225A, 225B of the tool elements 220A, 220B as the tool elements are held within the cartridge 200. FIG. 8B also shows the distal end 49 of the robotic arm 42 featuring pulley elements 120 and a raised or protruding boss element 130. The cartridge 200 is configured to permit entry of the distal end 49 of the robotic arm 42 through the access aperture 203 and into the arm and tool channel 202 of the cartridge body 201 to permit the distal end 49 of the robotic arm 42 to engage with the tool elements 220A, 220B, and, more specifically, for the boss element 130 to engage with the slots 230 of the tool elements 220A, 220B. The detents 212A, 212B are sized and positioned to engage with the notches 225A, 225B of the tool elements 220A, 220B when the tool elements 220A, 220B are held within the arm and tool channel 202 of the cartridge body 201 by the cradle portion 207. In some embodiments, the detents 212A, 212B have a circular or semicircular profile.

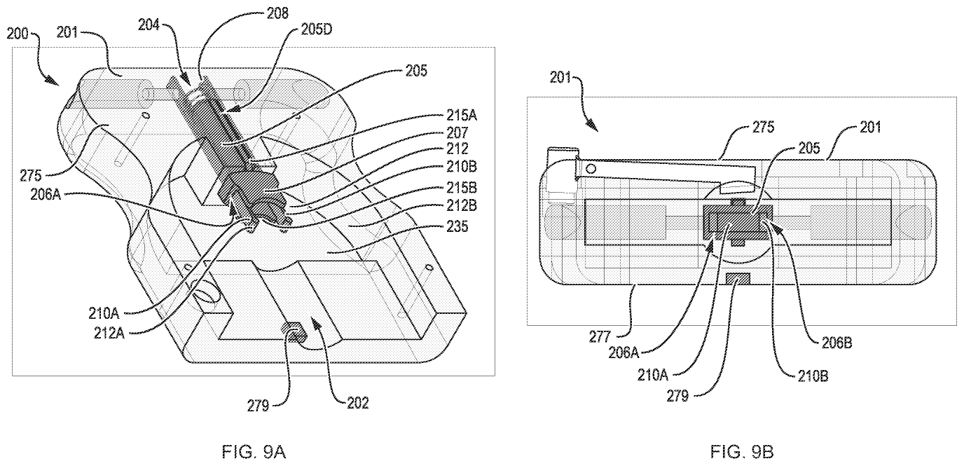

FIGS. 9A and 9B illustrate the cartridge 200 further including an RFID chip or tag 279 which is included in come embodiments. The cartridge 200 includes a cartridge body 201, which itself includes a cover portion 275 joined to a base portion 277. The cover portion is omitted from FIG. 9A for illustrative purposes. The cartridge body 201 also includes an arm or tool channel 202 configured to receive at least a portion of a distal end 49 of a robotic arm 42 and configured for withdrawal of the robotic arm 42 after tool elements (e.g., tool elements 80, 82) are installed on the distal end 49. A cradle portion 2207 is disposed at a first end of holder 205 and is configured to engage and hold tool elements. The cradle portion 2207 features an upper cradle portion part 215A and a lower cradle portion part 215B capable of holding the tool elements 80, 82. A second end portion of the holder 205D opposite the cradle portion 2207 extends into a holder channel 204 of the cartridge body 201 and is connected via a spring 208 to the cartridge body 201 either directly or through one or more different elements. The spring 208 and the holder channel 204 are configured to permit movement of the holder 205205 along a central axis of the holder channel 204 relative to the cartridge body 201 upon compression or extension of the spring.

The cartridge 200 also includes biasing elements 210A, 210B extending parallel to a central axis 209 of the holder channel 204. In some embodiments, each biasing element includes a detent 212A, 212B at a first end of the biasing element, which may be referred to as a proximal end of the biasing element. Each detent 212A, 212B is configured to engage by a corresponding notch on a tool element. Each biasing element 210A, 210B is connected to the cartridge body at second end, which may be described as a distal end in some embodiments. The holder 205 includes the biasing element channels 206A, 206B through which the biasing elements 210A, 210B extend. The biasing element channels 206A, 206B are configured to enable the holder 205 to move along the central axis 209 of the holder channel 204 with respect to the pair of biasing elements 210A, 210B while restricting lateral deflection of a portion of the biasing elements 210A, 210B positioned within the biasing element channels 206A, 206B.

The cartridge 200 also includes an RFID chip or tag 279. The RFID chip or tag 279 may be positioned near a surface of the arm and tool channel 202. The RFID chip or tag 279 is configured to interact with an RFID reader positioned on or in the distal end of the robotic arm that is able to read the contents of the RFID chip or tag 279. In some embodiments, the RFID chip or tag 279 includes or stores information regarding (e.g., tool elements 220A, 220B) stored in the cartridge. The information may include any or all of information identifying the tool elements (e.g., a type, a model number, etc.), information regarding an expiration date of the tool elements, a serial number associated with the tool elements, or other useful information. In some embodiments, when the RFID reader receives information from the RFID chip or tag 279 regarding the tool elements, a control system for the robotic arm may store the received information regarding tool elements currently mounted on the robotic arm, and/or may store an identification of tool elements currently mounted on the robotic arm based on the received information.

In some embodiments, after a first set of tool elements 220A, 220B are removed from the robotic arm 42 and the distal end 49 of the robotic arm 42 is inserted into a second cartridge holding a second set of tool elements, second information is received by the RFID reader of the robotic arm 42 from a RFID chip or tag 279 of the second cartridge regarding the second set of tool elements stored in the second cartridge. In some embodiments, the control system for the robotic arm 42 may store the received second information regarding the second set of tool elements currently mounted on the robotic arm 42. In response to receipt of the second information, the control system may overwrite an identification of the prior tool elements 220A, 220B as being currently mounted on the robotic arm 42 with an identification of the second set of tool elements as being currently mounted on the robotic arm based on the received second information. Additional aspects of an RFID tag or chip 279 of a cartridge 200 and an RFID reader of a distal end of a robotic arm are described below with respect to FIGS. 17-19. The operation of embodiments of RFID chip or tag 279 are further described below in the section entitled RFID Engagement Sensing.

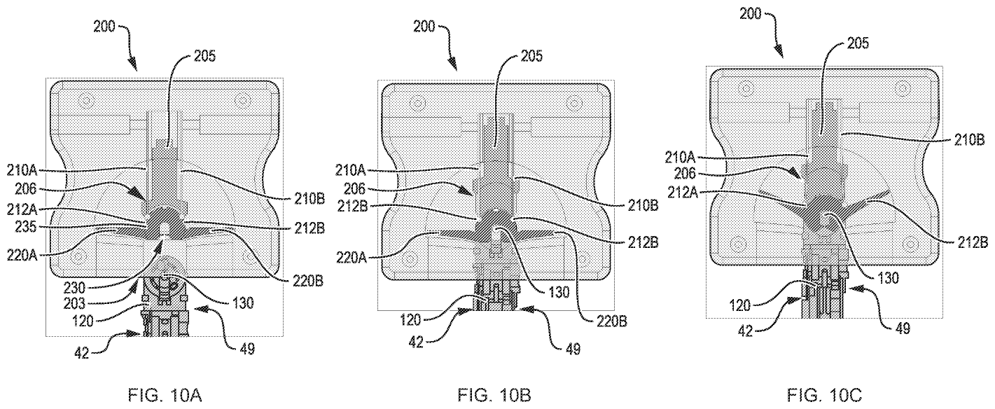

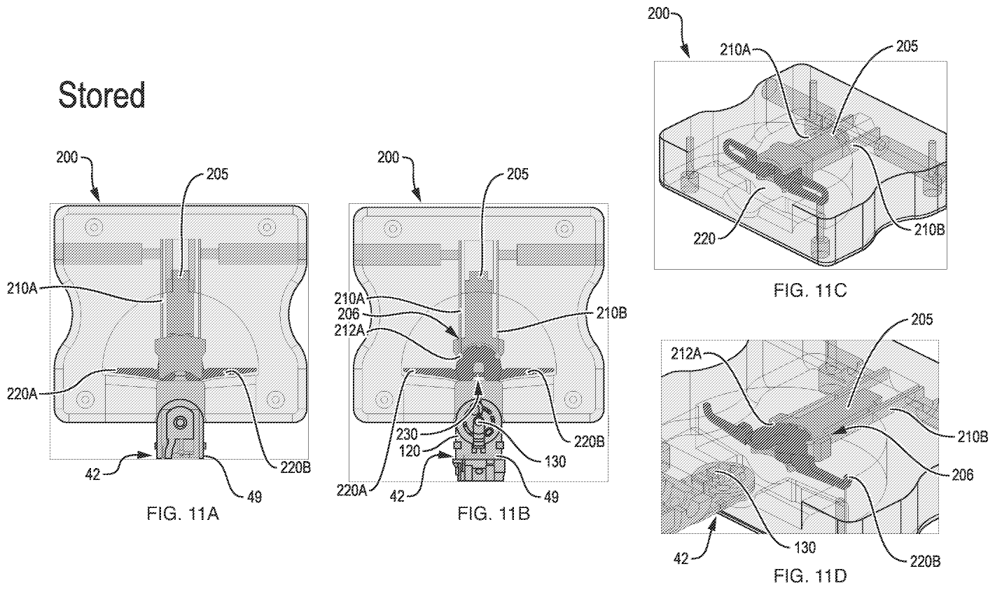

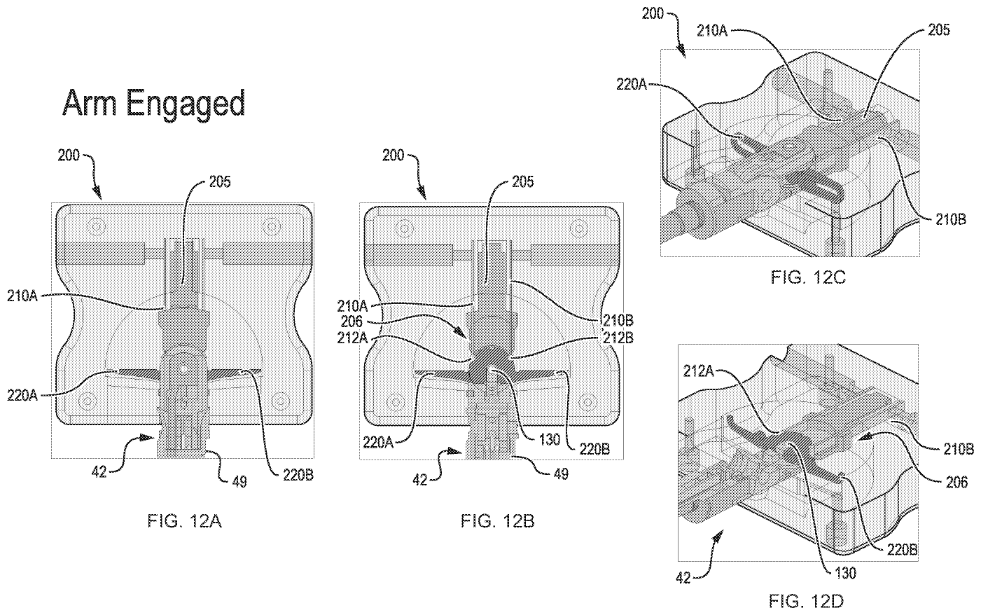

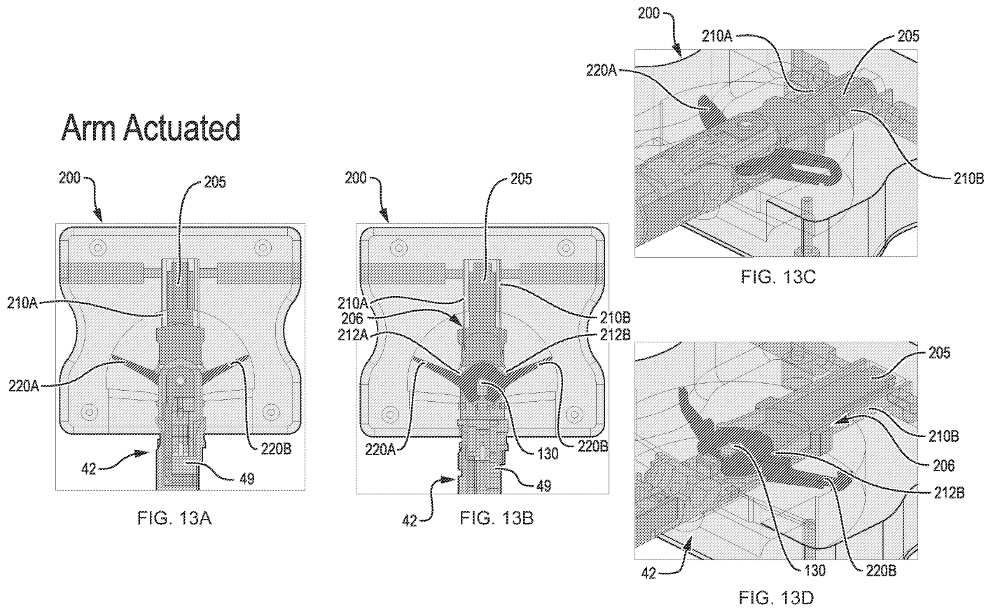

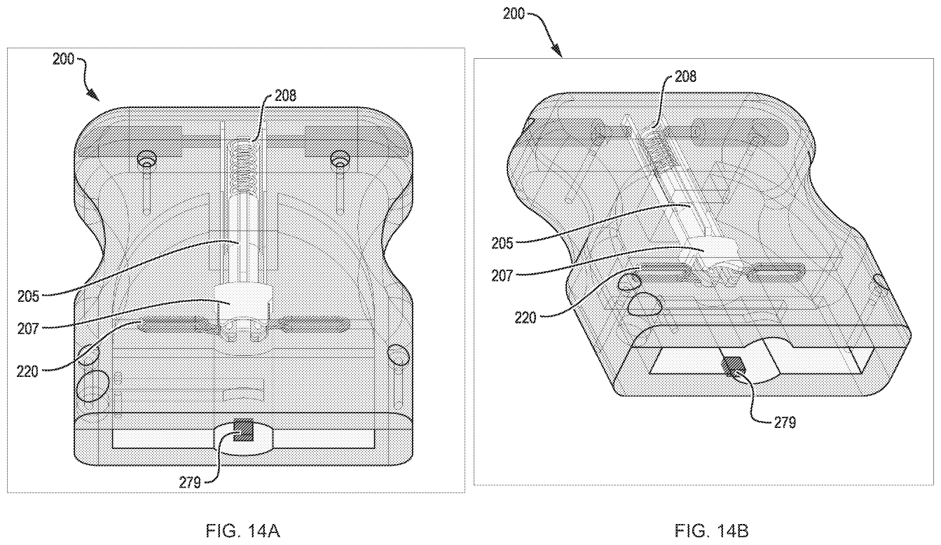

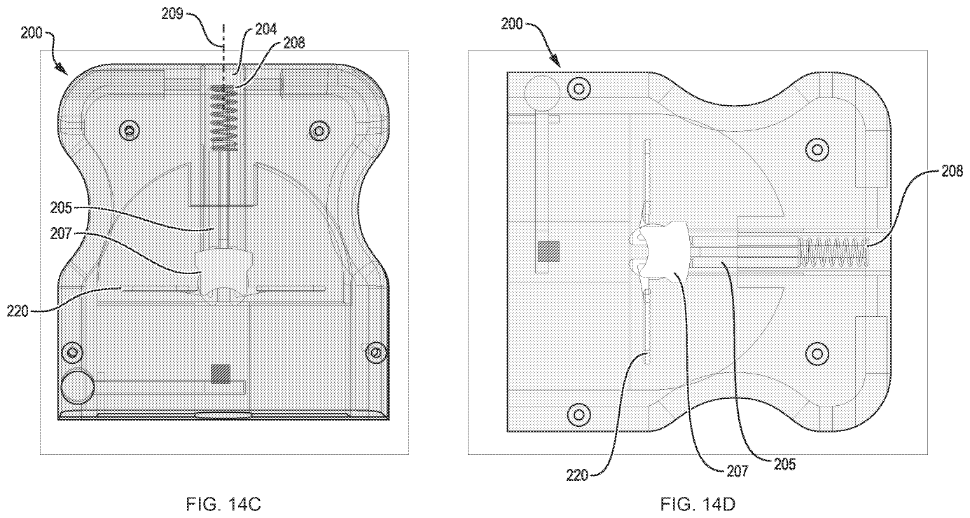

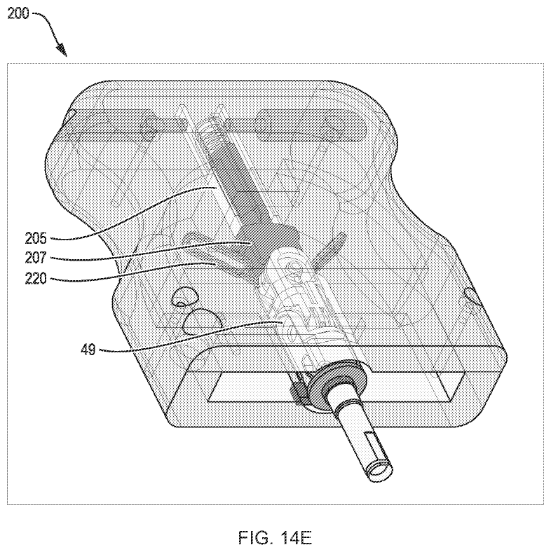

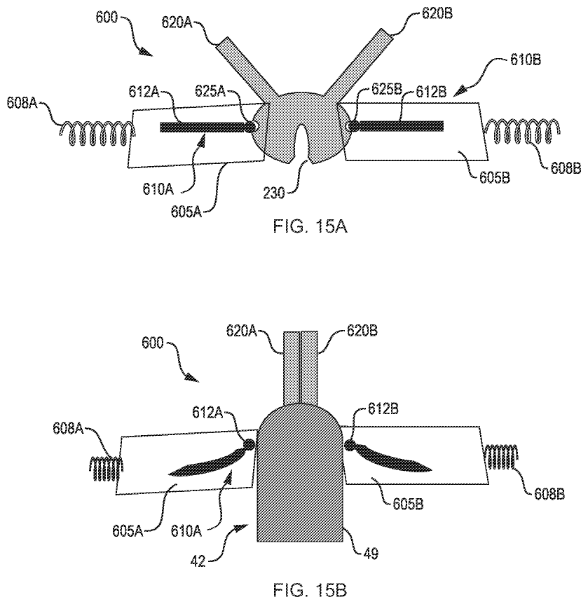

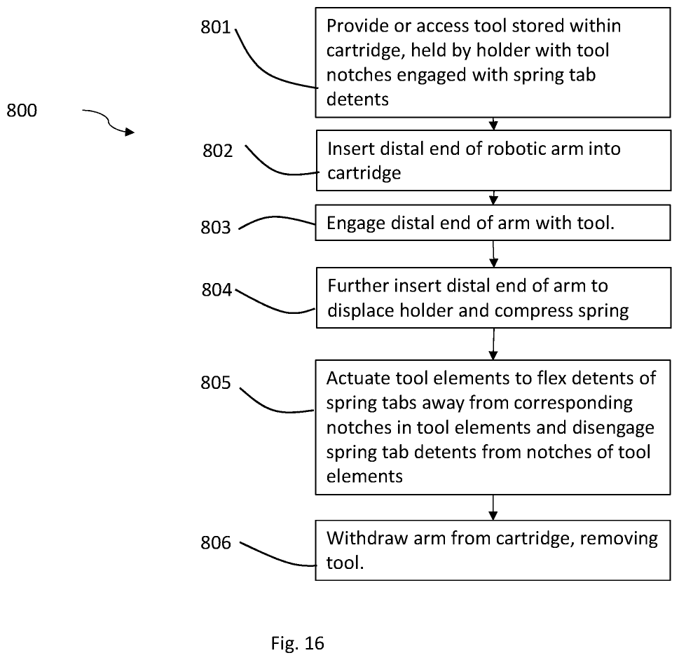

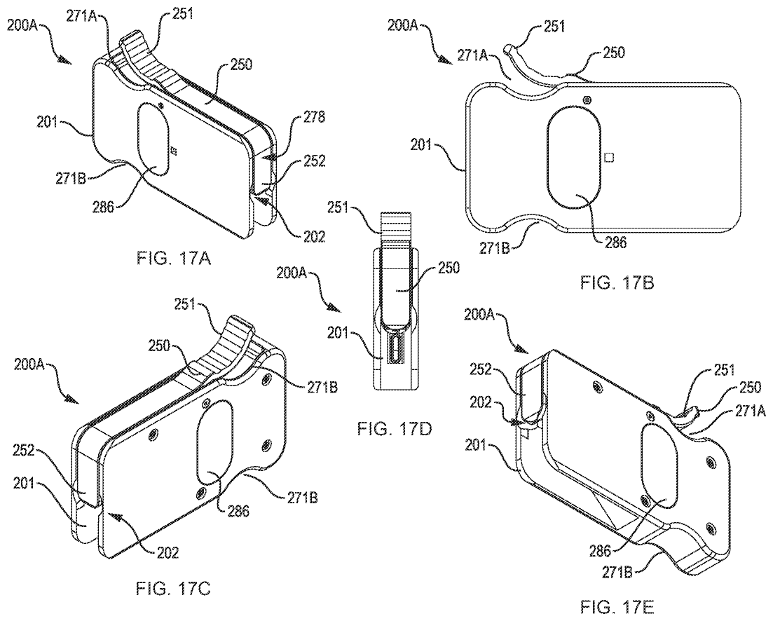

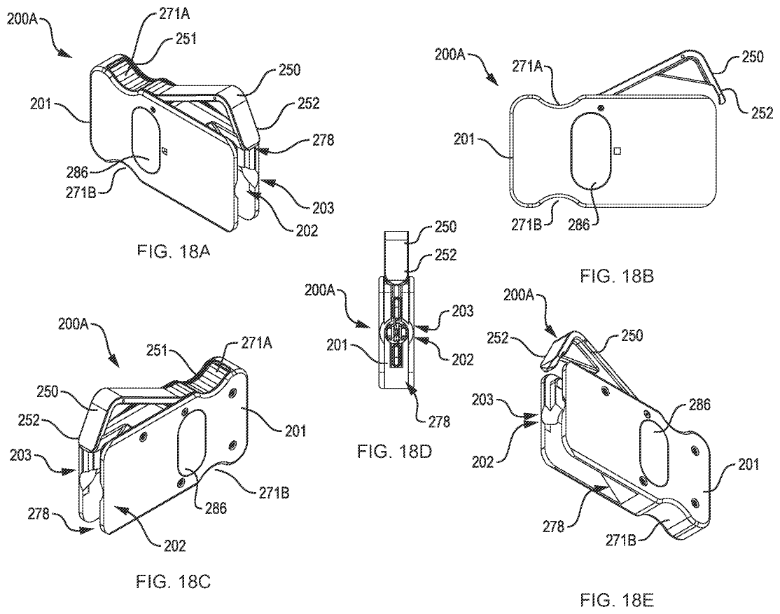

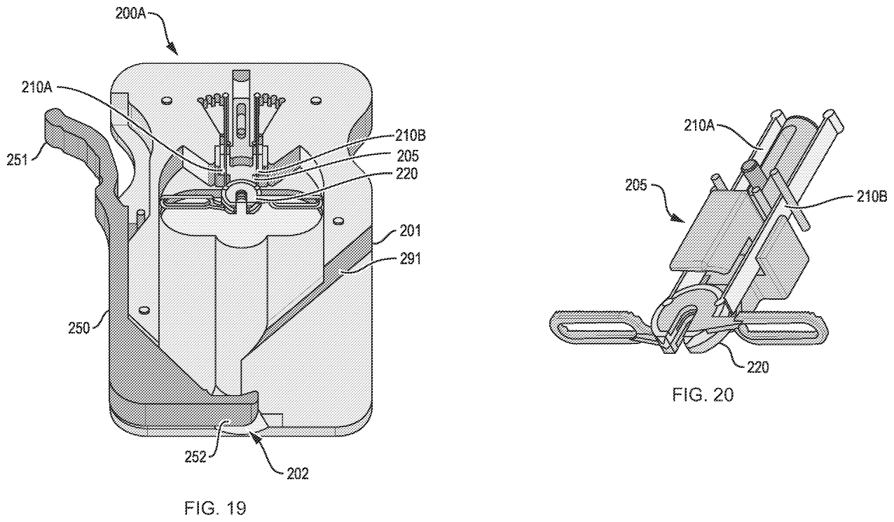

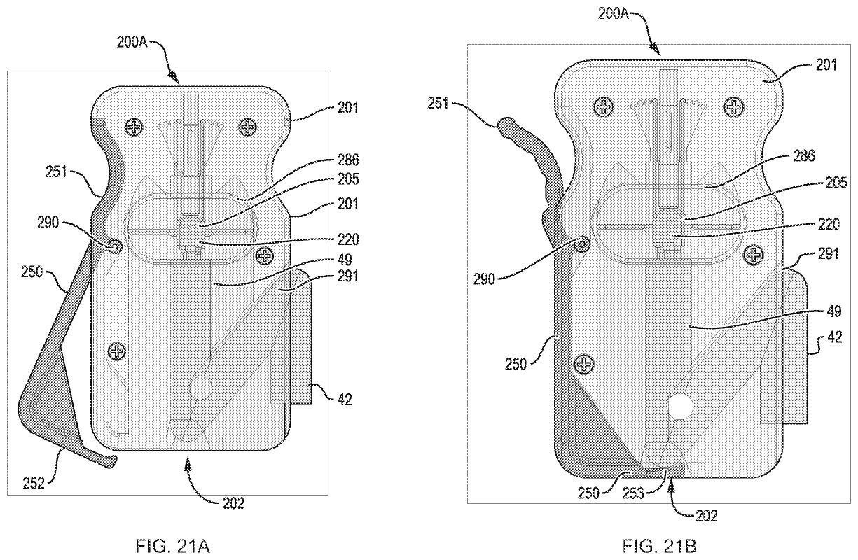

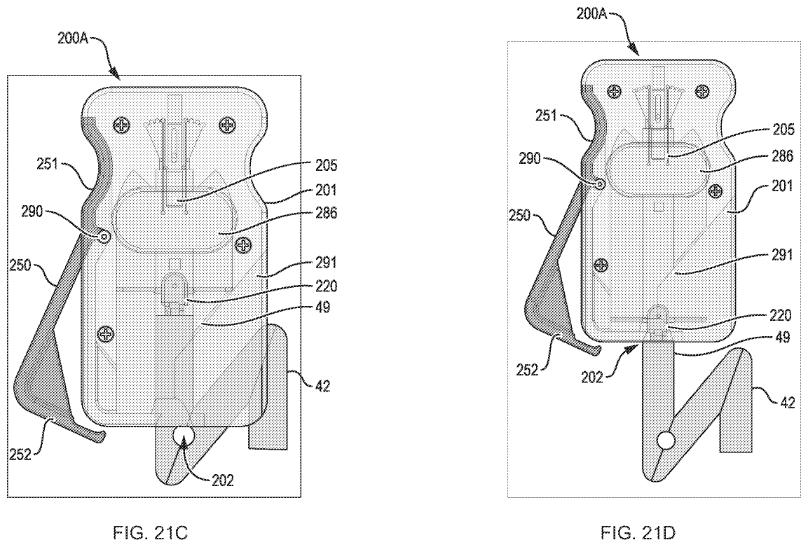

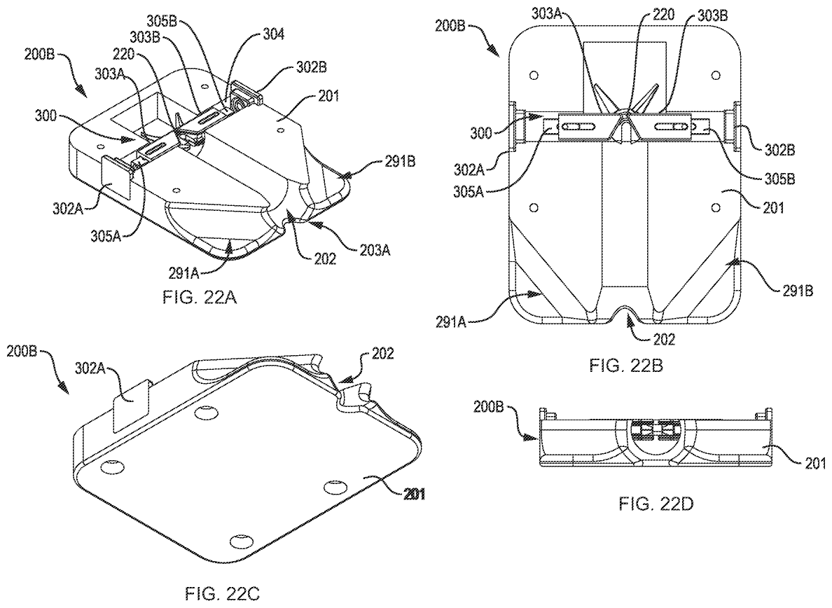

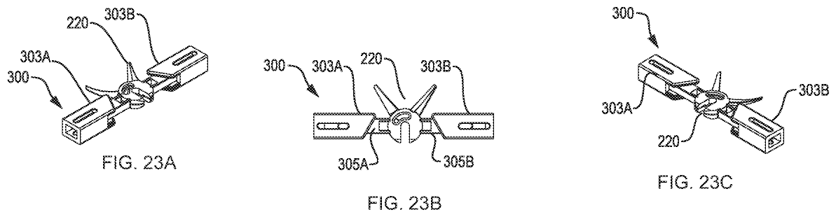

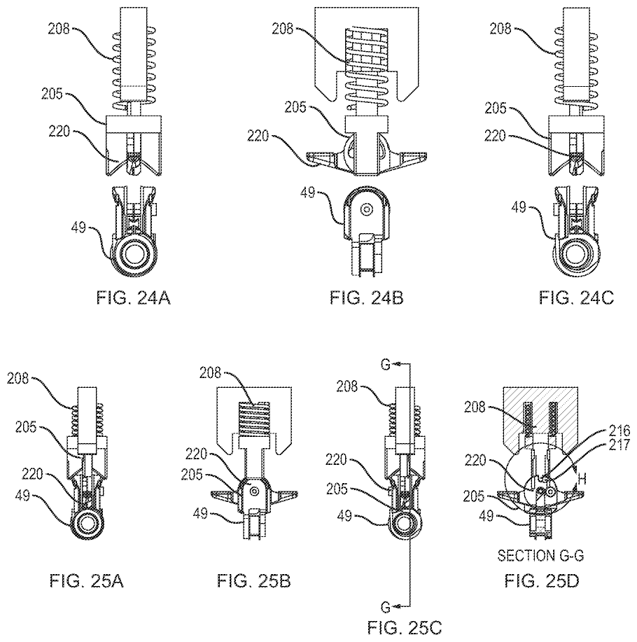

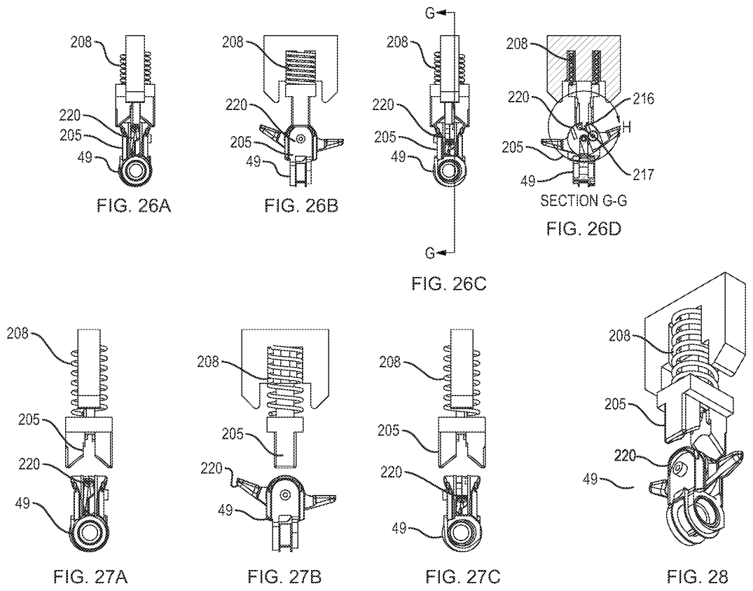

FIGS. 10A to 10C illustrate the process by which the distal end 49 of the robotic arm 42 engages with the tool elements 220A, 220B held within the tool holding cavity 235 of the cartridge 200. In FIG. 10A, the distal end 49 of the robotic arm 42 is inserted into the cartridge 200 via the tool channel 202 which includes a portion of the access aperture 203 in the side 278. The access aperture 203 in the side 278 may be outfitted with a reusable door or a single use covering material, such as a plastic sheet to provide protection from dust or other contaminates while still permitting entry of the distal end 49 of the robotic arm 42 as shown on FIG. 10A. FIG. 10B shows the further insertion of the distal end 49 of the robotic arm 42 such that the boss element 130 of the distal end 49 of the robotic arm 42 fits within the slot 230 of the tool elements 220A, 220B. As shown in FIG. 10C, the distal end 49 of the robotic arm 42 is now inserted further into the cartridge 200 pushing against the holder 205 to compress the spring 208 (not shown in FIG. 10C) and permit the holder 205 to move away from the opening. The holder 205 features two biasing element channels 206 that limit deflection of the biasing elements 210A, 210B. As the holder 205 recedes into the holder channel, portions of the biasing elements 210A, 210B proximate the detents 212A, 212B are released from the biasing element channels 206 of the holder 205 and are thus able to flex. As shown in FIG. 10C, the portion of each of the biasing elements 210A, 210B including detents 212A, 212B flexes outward due to the force from the body of tool elements 220A, 220B when they are no longer held in position by the holder 205. As the detents 212A, 212B on the biasing elements 210A, 210B deflect outwardly, they disengage from the corresponding notches 225A, 225B of the tool elements 220A, 220B thereby permitting the tool elements 220A, 220B to be withdrawn from the cradle portion 207 of the holder 205. After the distal end 49 of the robotic arm 42 has engaged the tool elements 220A, 220B and they have been released from the corresponding detents 212A, 212B, they may be withdrawn from the cartridge 201 body via the opening 270 of the cartridge body 201. In embodiments, the arm 42 may rotate the tool elements 222 to close the arms and reduce the width of the tool 220 to permit easier withdrawal through the opening 203.

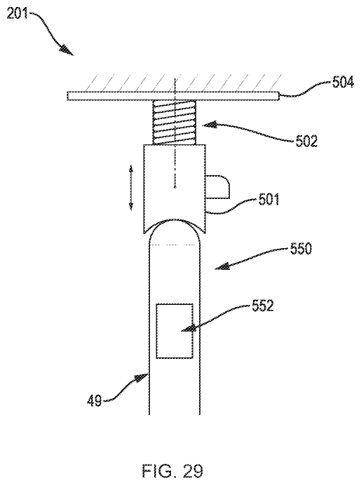

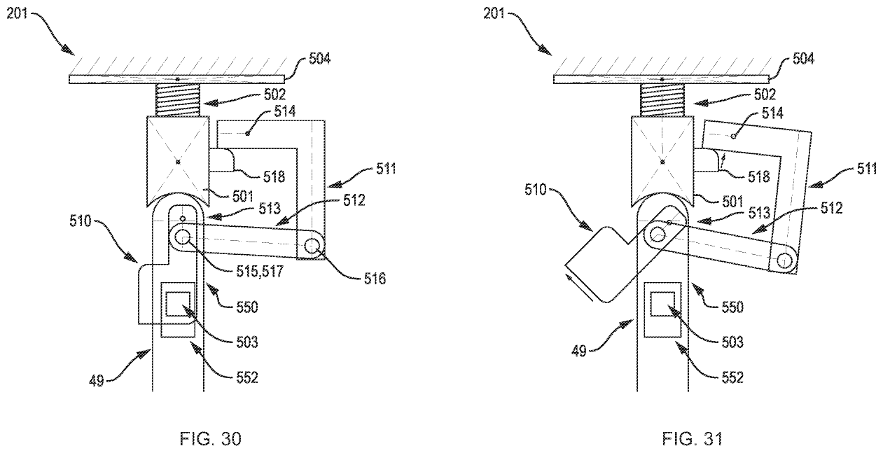

FIGS. 11A-11D further illustrate the cartridge 200 when the tool 220 is in the stored position within the cartridge 200. In particular, FIG. 11A shows a top view with the cover portion 275 of the cartridge body 201 omitted for illustrative purposes, showing among other things, the holder 205. FIG. 11B shows a top view in which the biasing element channels 206 of the holder 205 and the detents 212A, 212B are visible and the biasing elements 210A, 210B extend through the biasing element channels 206. FIG. 11C is a perspective view with the cover portion omitted for illustrative purposes, and FIG. 11D is a perspective view of a section through the cartridge.

FIGS. 12A-12D illustrate views of the cartridge 200 when the arm 42 is engaged with the tool 220 after the distal end 49 is inserted into the cartridge 200. FIG. 12A shows a top view with the cover portion 275 of the cartridge body 201 omitted for illustrative purposes, showing among other things, the holder 205. FIG. 12B shows a top view in which the biasing element channels 206 of the holder 205 and the detents 212A, 212B are visible and the biasing elements 210A, 210B extend through the biasing element channels 206. FIG. 12C is a perspective view with the cover portion omitted for illustrative purposes, and FIG. 12D is a perspective view of a section through the cartridge. FIGS. 12A-12D depict the configuration where the arm 42 is engaged, but force is not applied by the arm 42 to shift the holder 205.

FIGS. 13A-13D illustrate views of the cartridge 200 when the arm 42 is engaged with the tool 220 and has been actuated to apply force to the holder 205 and shift the holder 205 to compress the spring 208. FIG. 13A shows a top view with the cover portion 275 of the cartridge body 201 omitted for illustrative purposes, showing among other things, the holder 205. FIG. 13B shows a top view in which the biasing element channels 206 of the holder 205 and the detents 212A, 212B are visible and the biasing elements 210A, 210B extend through the biasing element channels 206. FIG. 13C is a perspective view with the cover portion omitted for illustrative purposes, and FIG. 13D is a perspective view of a section through the cartridge. Further, shifting the holder 205 in turn shifts the biasing element channels 206 enabling a proximal portion of biasing elements including the detents 212A, 212B to deflect when the force applied by the tool elements 220A, 220B overcomes the spring force of the biasing elements 210A, 210B to thereby disengaging the detents 212A, 212B from corresponding notches of the tool elements 220A, 220B.

FIGS. 14A-14D illustrate views of a cartridge 200 including tool elements 220 mounted within a cradle portion 207 of a holder 205. Cartridge 200 includes a holder channel 204 with a central axis 209. The cartridge 200 also includes an RFID tag 279 which may be an RFID chip configured to interact with an RFID reader 552 which may be an RFID antenna in the distal end 49 of the robotic arm 42 in accordance with some embodiments. FIG. 14E shows the cartridge 200 with the distal end 49 of the robotic arm 42, inserted into the cartridge 200 and engaged with the tool elements 220.

FIGS. 15A and 15B illustrate a cartridge 600 that includes two holders 605A, 605B, each connected to a spring 608A, 608B. In the cartridge 600, the holders 605A, 605B do not move parallel to an insertion direction, but instead move laterally perpendicular to the insertion direction. Ends of the holders 605A, 605B closest to the tool elements 620A, 620B are configured to support the tool elements 620A, 620B from the sides. In the cartridge 600, there are biasing elements 610A, 610B associated with each holder. In some embodiments, each biasing element 610, 610B includes a detent 612A, 612B that engages with corresponding retention mechanisms 625A, 625B on the tool elements 620A, 620B. In some embodiments, the retention mechanisms 625A, 625B are notches or other element to receive a portion of the biasing element. Each holder 605A, 605B extends above and below a main body of the tool elements 620A, 620B (similar to, for example, tool elements 102 and 104, as shown in FIG. 6) on opposite sides of the tool elements 620A, 620B to cradle and hold the tool elements 620A, 620B on each lateral side. Each holder 605A, 605B is connected to a cartridge body 201 by a respective spring 608A, 608B. The springs 608A, 608B are configured to permit the holders 605A, 605B to be displaced laterally away from the tool elements 620A, 620B. The holder portions that move laterally are described below with reference to FIGS. 22A-22D.

As illustrated in FIG. 15B, to mount or install elements tool elements 620A, 620B onto the distal end 49 of a robotic arm 42, the distal end 49 is inserted into the cartridge and engages with the tool elements 620A, 620B. The distal end 49 is then advanced further to push the tool elements 620A, 620B and the holders 605A, 605B such that the holders 605A, 605B shift laterally away from the tool elements 620A, 620B. Further, the biasing elements 610A, 610B are deflected, thereby flexing the detents 612A, 612B away from the tool elements 620A, 620B and out of the notches 625A, 625B. In this way, the tool elements 620A, 620B may be engaged by the distal end 49 and disengaged from the holders 605A, 605B and the detents 612A, 612B such that the tool elements 620A, 620B may be withdrawn from the cartridge 600.

A method 800 depicted in FIG. 16 shows an exemplary process according to the present disclosure. Some embodiments include providing or accessing a cartridge 200 storing a tool 220 comprising tool elements 220A, 220B (801). The tool 220 may be stored within the cartridge 200, for example for shipping and storage of the tool 220 in the cartridge 200 before a procedure, for preparation for a procedure, or as a secondary or alternate tool during a surgical procedure. The holder 205 holds the tool 220 and the two notches 225A, 225B within the tool 220 engage with detents 212A, 212B on the biasing elements 210A, 210B of the cartridge 200.

Some embodiments include inserting the distal end 49 of the robotic arm 42 into the cartridge 200 (802). Some embodiments include engaging the distal end 49 of the robotic arm 42 with a tool 220 (803). For example, a boss element 130 on the distal end 48 may engage with a slot 230 of the tool 220. The engagement may occur via friction between surfaces of the two components. Some embodiments include further inserting the distal end 48 of the arm 42 to displace the holder 205 and compress the spring 208 (804). As the holder 205 moves in this direction, the holder 205 moves away from the detents 212A, 212B located on the end of the biasing elements 210A, 210B, and permits movement of the biasing elements. Some embodiments include actuating the tool elements to flex detents of the biasing elements away from corresponding notches in the tool element (805). Some embodiments include withdrawing the arm from the cartridge and thus removing the tool (806). The arm may articulate the tool elements to close them reducing the width of the tool for withdrawal from the cartridge.

It is also contemplated in the present disclosure that the tool 220 may be replaced within the cartridge 200, substantially by reversing the procedure described for removing the tool 220 from the cartridge 200, for example in method 800. Replacing the tool 220 within the cartridge 200 may be performed, for example, during a procedure, when it is desired to switch the tool 220 being used on the mechanical arm 42 and to maintain the tool 220 within a sterile, protected environment while a second tool is in use, such that, optionally, the first tool 220 may be reattached to the arm 42 and a subsequent phase of the procedure for further use of the tool 220. Additionally, replacing the tool 220 within the cartridge 200 may permit easier handling of the tool 220, for example by maintaining a tool and any bodily fluids or other bodily matter they are on within the cartridge 200 to facilitate cleaning of the surgical robot after the procedure has been completed, or during the procedure as necessary.

The cartridge body 201 may be made of a plastic material, such as such as PET (polyethylene terephthalate), HDPE (high-density polyethylene), PVC (polyvinyl chloride), PP (polypropylene), or PS (polystyrene). Alternatively, the cartridge body and cartridge supports may be made of metals including steel, stainless steel, aluminum, nickel, copper, zinc, tin and alloys (including brass, nickel-chromium alloys, etc.).

The biasing elements 210A, 210B may be made from any material or materials with sufficient mechanical properties to hold the tool elements in place by engaging the detents of the biasing elements with the corresponding notches of the tool elements. In some embodiments, the biasing elements may be made of spring steel ranging from 0.02 to 0.03 inches thick by 3 mm height. Alternatively, they may be made of other metals including stainless steel, aluminum, nickel, copper, zinc, tin and alloys (including brass, nickel-chromium alloys, etc.). A variety of types of springs may be used, such as compression springs, expansion springs (e.g., mounted on one side to the side of the cartridge containing the opening), torsion springs (e.g., torsion springs housed within the supports), etc. Alternatively the springs and biasing elements may be made of a flexible plastic material, such as a natural or synthetic rubber, PET (polyethylene terephthalate), HDPE (high-density polyethylene), PVC (polyvinyl chloride), PP (polypropylene), or PS (polystyrene).

FIGS. 17A, 17B, 17C, 17D, and 17E illustrate the cartridge 200A which includes the cartridge body 201, the side 278, and a window 286. The side 278 includes the access aperture 203 providing access to the arm and tool channel 202. The arm and tool channel 202 is configured to receive the distal end 49 of the robotic arm 42 through the access aperture 203. The window 286 allows visual observation within the cartridge 200A and permitting a view of the tool 220 within the cartridge 200A when the tool 220 is present. A movable cartridge lever 250 forms a portion of the side 278. The cartridge lever 250 rotates about a rotation axis between an open and a closed position. The cartridge lever 250 includes a lever grip 251 and a channel cover 252. The channel cover 252 is positioned over at least a portion of the access aperture 203 at one end of the arm and tool channel 202 when the cartridge lever 250 is in a closed position, as shown in FIGS. 17A-17E. The cartridge 200A also includes cartridge body grips 271A, 271B which are shaped to facilitate being held in a hand of a user and to permit the user to depress the cartridge lever 250 via the lever grip 251 while holding the cartridge body 201 via one or more of the cartridge body grips 271A, 271B in order to optionally permit one-handed operation of the cartridge lever 250 so that the user may hold the cartridge 200A and depress the cartridge lever 250 with one hand. The lever grip 251 may be configured as a curved protrusion such that in the closed position the user may either grip cartridge body grip 271A or alternatively lever grip 251 whereas in an open position of the cartridge lever 250 (as shown in FIGS. 18A-18E), the lever grip 251 may be positioned essentially in line with or above the cartridge body grip 271A so that the user may grip the lever grip 251 with substantially the same gesture as the cartridge body grip 271A.

FIGS. 18A, 18B, 18C, 18D, and 18E illustrate the cartridge 200A with the cartridge lever 250 in the open position. The cartridge lever 250 may be moved to the open position by depressing the lever grip 251. When the lever grip 251 is depressed, the cartridge lever 250 pivots such that the channel cover 252 moves away from the arm and tool channel 202 to permit access to the arm and tool channel 202. With the cartridge lever 250 in the open position, the distal end 48 of the robotic arm 42 may be placed in the arm and tool channel 202 in order to access the tool 220. As shown in FIGS. 18A-18E, when the cartridge lever 250 is in the open position, the lever grip 251 shifts toward the cartridge body grip 271A.

FIG. 19 illustrates a cross-section of the cartridge 200A with the cartridge lever 250 in the closed position and showing the tool 220 held within the holder 205 by biasing elements 210A and 210B which engage with the tool 220. The cartridge body 201 also includes an access channel 291. FIG. 20 shows a detail of the holder 205 with the tool 220 held by the biasing elements 210A, 210B.

FIG. 21A illustrate a cross-section of the cartridge 200A with the cartridge body 201 and the cartridge lever 250 in the open position to allow access to the arm and tool channel 202 of the cartridge 200A. The cartridge body 201 also includes an access channel 291 which allows robotic arm 42 to access the cartridge 200A in a “V”-shaped configuration, as shown in FIGS. 21A and 21B. The cartridge 200A includes the window 286 allowing a view of the interior of the cartridge 200A showing the tool 220. The cartridge lever 250 includes the lever grip 251 and the channel cover 252. As shown in FIG. 21A, when the lever grip 251 is depressed, the cartridge lever 250 pivots on pivot point 290 moving the channel cover 252 away from the arm and tool channel 202 and allowing access to the arm and tool channel 202 by the distal end 49 of the robotic arm 42. The distal end 49 of the robotic arm 42 may engage the tool 200A.

FIG. 21B illustrates a cross-section of the cartridge 200A with the cartridge body 201 and the cartridge lever 250 in the closed position to prevent access to the arm and tool channel 202 of the cartridge 200A. The cartridge 200A includes the window 286 allowing a view of the interior of the cartridge 200A showing the tool 220. The cartridge lever 250 includes the lever grip 251 and the channel cover 252. As shown in FIG. 21B, when the lever grip 251 is released, the cartridge lever 250 pivots back on pivot point 290 moving the channel cover 252 over from the arm and tool channel 202 and covering the arm and tool channel 202 by the distal end 49 of the robotic arm 42. The cartridge lever 250 includes a curved portion 253 which mates with arm 42. The engagement between curved portion 253 and the arm 42 facilitates mating the cartridge 200A with the arm 42 so that the distal end 49 of arm 42 may engage the tool 220. The interaction between the curved portion 253 and the distal end 49 when the cartridge lever 250 is in the closed position may provide information to the user about the engagement of the arm 42 with the cartridge 200A. Closing cartridge lever 250 over the distal portion 49 provides confirmation that the distal portion 49 is fully inserted into the cartridge 200A when the distal portion 49 meets the curved portion 243.

Alternatively, if the cartridge lever 250 does not close over the distal portion 49, this provides an indication to the user that the distal portion 49 is not properly seated or the cartridge 200A is otherwise blocked. The user can also use the act of closing the cartridge lever 250 to facilitate seating the cartridge 200A on the arm 42 by using the force of the cartridge lever 250 against the arm 42 to help seat the cartridge 200A. Additionally, as shown in FIGS. 21A-21D, the arm 42 is positioned in a “V”-shaped configuration; that is, a configuration in which segments of the arm 42 form a “V”-shaped arrangement with a portion of the arm 42 arranged generally in the same plane as the distal end 49 and engaging along one of the access channels 291A, 291B.

FIG. 21C illustrates a cross-section of the cartridge 200A with the cartridge body 201 and the cartridge lever 250 in the open position to allow access to the arm and tool channel 202 of the cartridge 200A. As shown in FIG. 21C, when the lever grip 251 is depressed, the cartridge lever 250 again pivots on pivot point 290 moving the channel cover 252 away from the arm and tool channel 202 and allowing the robotic arm 42 to withdraw from the arm and tool channel 202, holding the tool 220.

FIG. 21D illustrates a cross-section of the cartridge 200A with the cartridge body 201 and the cartridge lever 250 in the open position to allow access to the arm and tool channel 202 of the cartridge 200A as the robotic arm 42 continues to withdraw from the arm and tool channel 202, holding the tool 220.

FIGS. 22A, 22B, 22C, and 22D illustrate the cartridge 200B with the cartridge body 201 including access channels 291A, 291B to facilitate access to the arm and tool channel 202. Similar to the embodiment described above with reference to FIGS. 15A-15B, the cartridge 200B includes a holder 300 that includes a first holder portion 303A with a first internal tab 305A and a second holder portion 303B with a second internal tab 305B that are positioned laterally with respect to the arm and tool channel 202 to hold the tool 220. In the cartridge 200B, the two holder portions 203A and 303B are positioned in a holder channel 304 extending generally perpendicularly to the arm and tool channel 202. A first housing segment 302A and a second housing segment 302B are positioned within the holder channel 304 at the housing 201. In some embodiments, large springs may be positioned between the first housing segment 302A and the first holder portion 303A and between the second housing segment 302B and the second holder portion 303B in order to bias the holder portions 303A, 303B inwardly to support the holder 220. Small springs may be positioned between the first internal tab 305A and the first holder portion 303A and between the second internal tab 305B and the second holder portion 303B. A pin (not shown) is anchored in the cartridge body 201 and passes through a slot in the first holder portion 303A and the first internal tab 305A and between the second holder portion 303B and the second internal tab 305B, respectively, thus constraining the motion of both sets of components. The access channels 291A, 291B allow the robotic arm 42 to access the cartridge 200B in a “V”-shaped configuration similar to that described in connection with FIGS. 21A and 21B, but permitting access from either side of the cartridge body 201. The cartridge 200B includes an access aperture 203A with a notched shape to provide access and facilitate directing and seating the arm 42 within the arm and tool channel 202.

FIGS. 23A, 23B, and 23C each show a detail of the holder 300 with the holder sections 303A, 303B holding the tool 220. The first internal tab 305A and the second internal tab 305B press into receiving mechanisms in the tool 220 to hold the tool 220 in place. The first holder portion 303A and the second holder portion 303B constrain the tool 220 on the top and bottom of tool 220 and assist in maintaining the tool 220 in position. The tool 220 is removed from the holder 300 by inserting the robotic arm 32, engaging the tool 220 then pushing the tool 220 in order to disengage the first internal tab 305A and the second internal tab 305B and release the tool 220.

FIGS. 24A-28 each show a detail view including the holder 205 with a tool 220 as the robotic arm 42 approaches the tool 220, then engages the tool 220, and finally withdraws the tool 220, as described below. In holder 205, the tool 220 is captured by using a protrusion 216 in the holder 205, which may be a cylindrical protrusion, that interfaces with a groove 217 in the tool 220 (see FIGS. 25D, 26D). When the tool 220 is open to approximately 180 degrees, the tool 220 may be locked to the holder 205 via the engagement of the protrusion 216 with the groove 217. When the tool 220 actuates, the groove 217 and the protrusion 216 may be disengaged, permitting removal of the tool 220 from the holder 205. Additionally, features/grooves on holder 205 prevent rotation of the tool 220 while it is being held by the holder 205, as shown in FIG. 26D, 28.

FIGS. 24A, 24B, and 24C each illustrate the spring 208 and the holder 205 holding the tool 220 as the distal end 49 of the robotic arm 42 approaches the tool 220. FIG. 24A illustrates a first side view of the holder 205 with spring 208 and tool 220 and a first side view of the distal portion 49 of robotic arm 42. FIG. 24B is a front view. FIG. 24C illustrates an opposite side view.

FIGS. 25A, 25B, 25C, and 25D illustrate the spring 208 and the holder 205 holding the tool 220 as the distal end 49 of the robotic arm 42 engages with the tool 220. FIG. 25A illustrates a first side view of the holder 205 with spring 208 and tool 220 and a first side view of the distal portion 49 of robotic arm 42. FIG. 25B is a front view. FIG. 25C illustrates an opposite side view. FIG. 25D illustrates a cross section G shown in FIG. 25C.

FIGS. 26A, 26B, 26C, and 26D illustrate the spring 208 and the holder 205 holding the tool 220 as the distal end 49 of the robotic arm 42 engages with the tool 220 and manipulates the tool 220. FIG. 26A illustrates a first side view of the holder 205 with spring 208 and tool 220 and a first side view of the distal portion 49 of robotic arm 42. FIG. 26B is a front view. FIG. 26C illustrates an opposite side view. FIG. 26D illustrates a cross section G shown in FIG. 25C.

FIGS. 27A, 27B, 27C, and 28 illustrate the spring 208 and the holder 205 as the distal end 49 of the robotic arm 42 withdraws the tool 220 from the holder 205. FIG. 27A illustrates a first side view of the holder 205 with spring 208 and tool 220 and a first side view of the distal portion 49 of robotic arm 42. FIG. 27B is a front view. FIG. 27C illustrates an opposite side view. FIG. 28 illustrates a perspective view of the spring 208 and the holder 205 as the distal end 49 of the robotic arm 42 withdraws the tool 220 from the holder 205. RFID Engagement Sensing

As explained above with respect to FIGS. 9A, 9B, and 14A-14E, in some embodiments, the RFID chip or tag 279 of a cartridge 200 may include or store information regarding tool elements (e.g., tool elements 220A, 220B) stored in the cartridge. The information may include any or all of information identifying the tool elements (e.g., a type, a model number, etc.), information regarding an expiration date of the tool elements, a serial number associated with the tool elements, or other useful information. In some embodiments, when the RFID reader receives information from the RFID chip or tag regarding the tool elements, a control system for the robotic arm may store the received information regarding tool elements currently mounted on the robotic arm, and/or may store an identification of tool elements currently mounted on the robotic arm based on the received information.

In some embodiments, after a first set of tool elements are removed from the robotic arm and the distal end of the robotic arm is inserted into a second cartridge holding different second tool elements, second information is received by the RFID reader of the robotic arm from a RFID chip or tag of the second cartridge regarding the different second tool elements stored in the second cartridge. In some embodiments, the control system for the robotic arm may store the received second information regarding the second tool elements currently mounted on the robotic arm. In response to receipt of the second information, the control system may overwrite an identification of the prior tool elements as being currently mounted on the robotic arm with an identification of the second tool elements as being currently mounted on the robotic arm based on the received second information.

In some embodiments, interactions between an RFID chip or tag of a cartridge and an RFID reader of a distal end of a robotic arm can also be used to determine a proximity of certain location on the distal end of robotic arm (e.g., the location of the RFID reader) to a desired corresponding location in an interior of the cartridge (e.g., the location of the RFID tag or chip when the distal end of the robotic arm has engaged the tool elements).

The present disclosure is also directed to cartridges that permit identification of the presence of a distal end of the robotic arm using radio-frequency identification (RFID) sensing, for example in order to confirm that the arm is engaged with a surgical tool within the cartridge. The RFID sensing may also be used to provide information regarding the tool stored in the cartridge. The RFID reader of the robotic arm and tag or chip of the cartridge may be used as a proximity sensor based upon whether or not the RFID reader is able to read the tag data. Some devices and methods permit proximity identification while the cartridge is unpowered and are relatively, simple while also greatly improving an ability of the RFID reader of the robotic arm to use the presence or absence of an RFID tag in the cartridge as an accurate position indicator.

A number of factors affect the read range of an RFID sensor, including variations in the RFID tag, reader antenna, and surrounding interference-inducing materials and geometry. Whether an RFID sensor in a distal end of a robotic arm can read or detect an RFID tag in a cartridge may not be an accurate indication of a position of the robotic arm relative to a desired position in the cartridge. The present disclosure provides devices and methods for achieving effective use of an RFID reader and tag for proximity identification in connection with a cartridge for a surgical tool. In some embodiments, the devices and methods disclosed herein also feature an RFID blocking flag and mechanical linkage to permit use of an RFID reading and tag for proximity identification. For example, in some embodiments, a cartridge includes an RFID blocking flag that interferes with signal transmission between the RFID reader and the RFID tag until the RFID blocking flag is repositioned when the robotic arm is correctly positioned in the cartridge.

FIG. 29 illustrates a portion of the interior features of the cartridge 200 in which a holder 501 is mounted on a spring 502, and the spring 502 is attached at an opposite end to an internal wall 504 of the cartridge body 201. As shown in FIG. 29, the distal end of arm 550 has been inserted into the cartridge 200. The distal end of the arm 550 features an RFID antenna 552. The characteristics of an RFID reader/tag system are such that when an RFID tag is positioned within the cartridge 200, the RFID antenna 552 may register the cartridge 200 as being proximate to the RFID antenna 552 even if the proximal end of the robotic arm is not positioned directly at a desired location within the cartridge 200, for example, a location at which the proximal end engages with a tool and shifts the holder 501.

Some embodiments are provided with a means for blocking and unblocking the RFID tag 503 when the distal end 49 of the robotic arm 42 is inserted into the cartridge 200. Specifically, the RFID tag 503 may be unblocked when the distal end 49 is inserted into the cartridge 200, and is seated proximate the tool 220 in the holder 501, or impinges against holder 501. Blocking/unblocking the RFID tag 503 may permit an RFID reader 552 of the distal end 49 of the robotic arm 42 to read the RFID tag 503 when the distal end 49 of the robotic arm 42 has been inserted and has engaged the means for unblocking the RFID tag 503 such that the ability of the RFID reader 552 to recognize the presence of and read the RFID tag 503 also indicates that the distal end 49 is inserted and, preferably, fully inserted/engaged with the holder 501. Alternatively, failure of the RFID reader 552 to recognize an RFID tag 503 may be used to initiate a prompt to the user that the distal end 49 is not engaged properly with the cartridge 200. The means for blocking and unblocking the RFID tag 503 may be a mechanical means. In some embodiments, the present disclosure provides for an RFID blocking flag, that can be positioned within the cartridge 200 between an RFID tag 503 mounted on or in the cartridge 200 and a location at which an RFID reader 552 of a distal end 49 of a robotic arm 42 would be located when the distal end 49 of the robotic arm 42 is inserted in the cartridge 200. The RFID blocking flag 510 positioned at this blocking position would block or reduce a signal passing to the RFID tag or from the RFID tag when the RFID. The cartridge may be configured for repositioning the blocking flag out of the blocking position, for example, when the holder of the cartridge is retracted following application of force from the robotic arm. For example, this retraction of the holder may occur during step 804 of method 800 as described above.

An embodiment of a portion of a cartridge featuring an RFID blocking flag and assembly is depicted in FIGS. 18 and 19. In FIG. 30, the cartridge 200 includes an RFID tag 503 mounted within cartridge 200 that is and covered/blocked by an RFID blocking flag 510 in a blocking position. The RFID blocking flag 510 is formed of a material that blocks or reduces transmission of an RFID signal through the RFID blocking flag 510. When the RFID blocking flag is in a blocking position, the RFID blocking flag 510 blocks a signal to or from the RFID tag 503. In some embodiments, the RFID blocking flag 510 is connected to the cartridge at a first a fixed pivot 513 about which the RFID blocking flag 510 may rotate. Additionally, in some embodiments, the RFID blocking flag 510 is connected to the linkage bar 512 via the first axle 515. The first axle 515 is connected to a first linkage bar 512 at a first end of the first linkage bar 512. At a second end, the first linkage bar 512 is connected to a second linkage bar 511 via the second axle 516. Additionally, the first linkage bar 512 may pivot about the pivot point 517. A first axle 515 and a second axle 516 permit the RFID blocking flag 510, the first linkage bar 512, and the second linkage bar 511 to rotate relative to each other. The second linkage bar 511 has a first end connected to the first linkage bar 512 via the second axle 516 and a second end, opposite the first end, that is positioned so as to be impinged and displaced by a protruding tab 520 of the holder 501 when the holder 501 is shifted.

Additionally, the second linkage bar 511 features a fixed pivot point 514 about which the second linkage bar 511 may rotate. When the holder 501 is moved closer to the internal wall 504 of the cartridge body 201 and the spring 502 is compressed, the protruding tab 518 pushes against the second end of linkage bar 511 causing the second linkage bar 511 to rotate about pivot 514 and to cause the second linkage bar 511 to apply force via the second axle 516 to the first linkage bar 512, in turn causing the first linkage bar 512 to pivot about the fixed pivot 517 and, in turn, to apply force to the RFID blocking flag 510 via the first axle 515, causing the RFID blocking flag 510 to pivot about the fixed pivot 513 and causing the RFID blocking flag 510 to shift to a second position that is away from the blocking position with respect to the RFID tag 503. FIG. 31 depicts the second position of RFID blocking flag 510.

As shown in FIG. 31, once the RFID blocking flag 510 has shifted out of its initial, blocking position over the RFID tag 503, the RFID tag 503 is no longer blocked and the RFID antenna 552 may register the presence of the RFID tag 503. As a result, the movement of RFID blocking flag 510 causes RFID tag 503 to be registered by RFID antenna 552, and thereby the system recognizes that the distal end 49 of robotic arm 42 is positioned in such a way as to move RFID blocking flag 510. A person of ordinary skill in the art will appreciate that alternative arrangements of mechanical linkages may be used to achieve the result of displacing an RFID blocking flag, including the use of different positions of linkages, or alternative mechanical structures.

While some embodiments of the present invention have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

We claim:

1. A cartridge for securing one or more tool elements of a surgical tool and releasing the tool elements when engaged by a distal end of a robotic arm, the cartridge comprising:
a cartridge body defining a tool holding cavity, a holder channel, an arm and tool channel, and an access aperture, the arm and tool channel extending from the tool holding cavity to the aperture on a first side of the cartridge body and configured to receive a distal end of a robotic arm, and a holder within the tool holding cavity and configured to releasably secure the one or more tool elements.

2. The cartridge of claim 1, wherein the holder includes;
a cradle portion configured to engage at least a portion of one or more tool elements;
an end portion opposite the cradle portion, the end portion extending into the holder channel; and
a biasing member disposed in the holder channel, the biasing member having a first end connected to the end portion of the holder and having a second end connected to the cartridge body and configured to permit movement of the holder relative to the cartridge body upon compression or extension of the biasing member.

3. The cartridge of claim 2, further comprising:
a pair of biasing elements, each biasing element including a retention mechanism disposed at a proximal end of the biasing element and each biasing element secured to the cartridge body at a distal end, each retention mechanism configured to engage a corresponding receiving mechanism in a tool element body of a corresponding tool element.

4. The cartridge of claim 3, wherein the holder defines a pair of biasing element channels, each of the pair of biasing elements extending through a corresponding one of the biasing element channels, the biasing element channels configured to enable the holder to move along a central axis of the holder channel with respect to the pair of biasing elements while restricting lateral deflection of a portion of the biasing elements positioned within the biasing element channels.

5. The cartridge of claim 4, wherein the pair of biasing element channels are configured to hold the proximal ends of the biasing elements in a first position when the spring is in an extended position and to enable the proximal ends of the biasing elements to flex outwardly when the spring is in a compressed position.

6. The cartridge of claim 2, further comprising a cartridge lever forming at least in part an exterior side of the cartridge and movable between a closed position and an open position.

7. The cartridge of claim 6, wherein when the cartridge lever is in the closed position, the cartridge lever substantially blocks access to the cavity of the cartridge body.

8. The cartridge of claim 6, wherein the cartridge lever comprising a channel cover to cover the arm and tool channel when the cartridge lever is in the closed position.

9. The cartridge of claim 6, wherein the cartridge lever comprises a lever grip manipulable to move the cartridge lever between the closed position and the open position.

10. The cartridge of claim 3, further comprising one or more access channels to guide the distal end of the robotic arm to the arm and tool channel.

11. The cartridge of claim 1, wherein the cartridge further comprises an RFID tag.

12. The cartridge of claim 11, wherein the cartridge further comprises
an RFID blocking flag moveable between a blocking position and a non-blocking position; and
at least one mechanical linkage connected to the RFID blocking flag to shift the RFID blocking flag between the blocking position and the non-blocking position.

13. The cartridge of claim 1, wherein the holder comprises a protrusion configured to engage with a groove in a tool.

14. The cartridge of claim 13, wherein the protrusion is a cylindrical protrusion.

15. A surgical robotic system comprising:
the cartridge of claim 1; and
a robotic arm comprising a distal end configured to hold a surgical tool.

16. The surgical robotic system of claim 15, wherein the cartridge comprises an RFID tag and the robotic arm comprises an RFID reader.

17. A method for providing a surgical tool to a surgical robotic system, the method comprising:
providing a cartridge for holding a surgical tool therein;
inserting a distal end of a robotic arm of the surgical robotic system into the cartridge;
engaging the surgical tool using the distal end of the robotic arm;
releasing the surgical tool from the holder; and
withdrawing the surgical tool from the cartridge.

18. The method of claim 17, wherein releasing the surgical tool from the holder comprises actuating tool elements of the surgical tool to flex retention mechanisms of biasing elements of the holder of the cartridge away from corresponding receiving mechanisms in the surgical tool.

19. The method of claim 17, wherein withdrawing the surgical tool comprises rotating or closing the surgical tool via the robotic arm to release the surgical tool from the holder of the cartridge.

20. The method of claim 17, further comprising pivoting a cartridge lever of the cartridge in order to allow access to the cartridge by the robotic arm.
